(19) United States
(12) Patent Application Publication
(10) Pub. No.: US 2021/0046044 A1
(43) Pub. Date: Feb. 18, 2021

(54) USE OF (+)-1-(3,4-DICHLOROPHENYL)-3-AZABICYCLO[3.1.0]HEXANE TO TREAT ADDICTIVE AND ALCOHOL-RELATED DISORDERS

(71) Applicants: Anthony Alexander McKinney, Newton Center, MA (US); Franklin Porter Bymaster, Brownsburg, IN (US)

(72) Inventors: Anthony Alexander McKinney, Newton Center, MA (US); Franklin Porter Bymaster, Brownsburg, IN (US)

(21) Appl. No.: 16/865,903

(22) Filed: May 4, 2020

Related U.S. Application Data
(63) Continuation of application No. 15/813,703, filed on Nov. 15, 2017, now abandoned.
Application No. 15/813,703 is a continuation of application No. 15/257,731, filed on Sep. 6, 2016, now abandoned.
Application No. 15/257,731 is a continuation of application No. 14/170,290, filed on Jan. 31, 2014, now abandoned.
Application No. 14/170,290 is a continuation of application No. 13/507,610, filed on Jul. 13, 2012, now abandoned.
Application No. 13/507,610 is a continuation of application No. 13/335,981, filed on Dec. 23, 2011, now abandoned.

(51) Int. Cl. A61K 31/403; A61K 45/06

(52) U.S. Cl. CPC A61K 31/403; A61K 45/06

(57) ABSTRACT

The present invention relates to (+)-1-(3,4-dichlorophenyl)-3-azabicyclo[3.1.0]hexane and pharmaceutically acceptable salts thereof, compositions comprising (+)-1-(3,4-dichlorophenyl)-3-azabicyclo[3.1.0]hexane or a pharmaceutically acceptable salt thereof, and methods for treating or preventing an alcohol-related or addictive disorder. The (+)-1-(3,4-dichlorophenyl)-3-azabicyclo[3.1.0]hexane or pharmaceutically acceptable salt thereof is preferably substantially free of its corresponding (−)-enantiomer

### RELATED APPLICATIONS

This application claims priority benefit of U.S. Continuation patent application Ser. No. 13/297,452, filed Nov. 16, 2011, the disclosure of which is incorporated herein in its entirety by reference.

### TECHNICAL FIELD

The present invention relates to use of (+)-1-(3,4-dichlorophenyl)-3-azabicyclo[3.1.0]hexane or a pharmaceutically acceptable salt thereof to treat or prevent an addictive or alcohol-related disorder comprising administering to a patient (+)-1-(3,4-dichlorophenyl)-3-azabicyclo[3.1.0]hexane or a pharmaceutically acceptable salt thereof.

### BACKGROUND OF THE INVENTION

Abuse and addiction to alcohol, nicotine, and illegal substances represent a significant public health concern at a cost upwards of half a trillion dollars a year in the United States in view of the combined medical, economic, criminal, and social impact (The Science of Addiction, NIH Pub. No 10-5605, August, 2010). In the United States, abuse of drugs and alcohol is estimated to contribute to the the death of more than 100,000 people, while tobacco is associated with an estimated 440,000 annual deaths (The Science of Addiction, NIH Pub. No 10-5605, August, 2010).

The addictiveness of certain drugs and compulsive behaviors is linked to the excitation of dopamine mediated reinforcement/reward pathways in the central nervous system (Abbott, 2002); Montague et al., 2004). Dopamine is a monoamine neurotransmitter that plays a critical role in the function of the hypothalamic-pituitary-adrenal axis and in the integration of information in sensory, limbic, and motor systems. The mesolimbic dopamine pathway, in which dopamine cells in ventral tegmental area project into nucleus accumbens, appears to be critical for drug reward (Wise, 2009). Other dopamine pathways, including mesostriatal (dopamine cells in substantia nigra projecting into dorsal striatum) and mesocortical (dopamine cells in ventral tegmental area projecting into frontal cortex) are now also recognized to contribute to drug reward and addiction (Wise, 2009). The mode of dopamine cell firing also differently modulates the rewarding and conditioning effects, of drugs (predominantly phasic dopamine cell firing) compared to the changes in executive function that occur in addiction (predominantly tonic dopamine cell firing) (Wanat et al., 2009; Grace, 2000).

The primary mechanism for termination of dopamine neurotransmission is through reuptake of released dopamine by Na⁺/Cl⁻ dependent plasma membrane transporters (Hoffman et al., 1998, Front. Neuroendocrinol. 19(3):187-231). Depending on the surrounding ionic conditions, the dopamine transporter can function as a mediator of both inward directed dopamine transport (i.e., “reuptake”) and outward directed dopamine transport (i.e., “release”). The functional significance of the dopamine transporter is its regulation of dopamine neurotransmission by terminating the action of dopamine in a synapse via reuptake (Hitri et al., 1994, Clin. Pharmacol. 17:1-22).

The dopamine transporter has long been a target for pharmacological treatment of abuse and/or addictive behavior. Targeting the dopamine transporter with ligands is largely based on the hypothesis that the ligand/drug would be expected to partially substitute for the abused substance (or behavior), thus decreasing self-administration of the substance and minimizing the craving for the substance. Along these lines, the dopamine/norepinephrine uptake inhibitor bupropion has been shown to be useful in nicotine addiction and has been approved for this use (Hays et al., 2003). Bupropion has likewise been shown to be useful in treating methamphetamine and other stimulant abuse (Elkashef et al., 2008).

Despite the advances made by targetting the dopamine transporter, there remains a need for effective treatment of abuse and addiction to alcohol, nicotine, and illicit or misused drugs, as well as addictive behaviors such as gambling and sexual addictions.

As described herein, a triple reuptake inhibitor that preferentially inhibits serotonin, then norepinephrine, and to a lesser extent, dopamine, has surprisingly been found to be effective in treating addictive and alcohol-related disorders.

### SUMMARY OF EXEMPLARY EMBODIMENTS

The present invention provides compositions comprising an effective amount of (+)-1-(3,4-dichlorophenyl)-3-azabicyclo[3.1.0]hexane or a pharmaceutically acceptable salt thereof. The present compositions can additionally comprise a pharmaceutically acceptable vehicle. These compositions are useful for treating or preventing an alcohol-related or addictive disorder.

The present invention also provides pharmaceutical compositions for treating or preventing ethanol consumption in a patient comprising an effective amount of (+)-1-(3,4-dichlorophenyl)-3-azabicyclo[3.1.0]hexane or a pharmaceutically acceptable salt thereof.

The present invention additionally provides pharmaceutical compositions for treating or preventing ethanol consumption and depression in a patient comprising an effective amount of (+)-1-(3,4-dichlorophenyl)-3-azabicyclo[3.1.0]hexane or a pharmaceutically acceptable salt thereof.

In another embodiment, the invention provides a method for treating or preventing ethanol consumption, comprising administering to a patient in need of such treatment or prevention an effective amount of (−)1-(3,4-dichlorophenyl)-3-azabicyclo[3.1.0]hexane or a pharmaceutically acceptable salt thereof.

Preferably, (+)-1-(3,4-dichlorophenyl)-3-azabicyclo[3.1.0]hexane or a pharmaceutically acceptable salt thereof, particularly when used in the present methods or compositions, is substantially free of its corresponding (−)-enantiomer. In a preferred embodiment, (+)-1-(3,4-dichlorophenyl)-3-azabicyclo[3.1.0]hexane or a pharmaceutically acceptable salt thereof substantially free of its corresponding (−)-enantiomer is used to treat or prevent an alcohol-related or addictive disorder.

Additional background information pertaining to (+)-1-(3,4-dichlorophenyl)-3-azabicyclo[3.1.0]hexane may be found, for example, in U.S. Pat. Nos. 4,435,419, 6,372,919, 7,098,229, U.S. patent application Ser. Nos. 11/205,956, 11/493,431, 11/740,667, 11/936,016, 12/135,053, 12/208,284, 12/334,432, 12/428,399, 12/782,705, 12/895,788, 13/048,852 and U.S. Provisional Patent Application No. 61/419,769, each of which is incorporated herein by reference in their entirety.

The present invention may be understood more fully by reference to the detailed description and examples which are intended to exemplify non-limiting embodiments of the invention.

### BRIEF DESCRIPTION OF THE DRAWINGS

FIGS. 1A and 1B have graphs showing the effects of (+)-1-(3,4-dichlorophenyl)-3-azabicyclo[3.1.0]hexane (DOV 21,947) in a binge operant responding assay of P rats. FIG. 1A shows the effect of (+)-1-(3,4-dichlorophenyl)-3-azabicyclo[3.1.0]hexane on ethanol consumption by P rats, while FIG. 1B shows its effect on sucrose consumption.

FIG. 2 has graphs showing the effects of (+)-1-(3,4-dichlorophenyl)-3-azabicyclo[3.1.0]hexane in a binge operant responding assay of HAP mice, with the top graph showing the effects on alcohol consumption and the bottom graph showing the effects on sucrose consumption.

### DETAILED DESCRIPTION OF EXEMPLARY EMBODIMENTS

The present invention provides methods for treating or preventing a wide variety of alcohol-related and addictive disorders. Using the methods of the invention, such disorders are amenable to treatment, prophylaxis, and/or alleviation of the disorder and/or associated symptom(s) by inhibiting reuptake of multiple biogenic amines causally linked to the targeted disorder. As shown herein, the triple reuptake inhibitor (+)-1-(3,4-dichlorophenyl)-3-azabicyclo[3.1.0]hexane was efficacious in treating alcohol consumption in rat and mouse models. This triple reuptake inhibitor has an unbalanced serotonin-norepinephrine-dopamine in vitro reuptake inhibition ratio of ˜1:2:8, respectively (Skolnick et al., 2003).

Given the well-developed association of the dopamine transporter with alcohol-related and addictive disorders, the efficacy of (+)-1-(3,4-dichlorophenyl)-3-azabicyclo[3.1.0]hexane for treating alcohol consumption was surprising since this compound more strongly inhibits serotonin and norepinephrine reuptake than dopamine reuptake.

Example I provides evidence in an alcohol-preferring rat (P rat) model indicating the efficacy of (+)-1-(3,4-dichlorophenyl)-3-azabicyclo[3.1.0]hexane for treating or preventing excess alcohol consumption. In that binge operant responding experiment, oral administration of (+)-1-(3,4-dichlorophenyl)-3-azabicyclo[3.1.0]hexane to P rats resulted in a decrease in alcohol consumption that was dose-dependent. Likewise, Example II provides evidence in a high-alcohol preferring (HAP) mouse model indicating the efficacy of (+)-1-(3,4-dichlorophenyl)-3-azabicyclo[3.1.0]hexane for treating or preventing excess alcohol consumption. In the binge operant responding experiment described in Example II, administration of (+)-1-(3,4-dichlorophenyl)-3-azabicyclo[3.1.0]hexane via injection to HAP mice resulted in a dose-dependent decrease in alcohol consumption. The efficacy of (+)-1-(3,4-dichlorophenyl)-3-azabicyclo[3.1.0]hexane in these well-accepted animal models of alcohol consumption indicates that (+)-1-(3,4-dichlorophenyl)-3-azabicyclo[3.1.0]hexane will likewise be efficacious in humans.

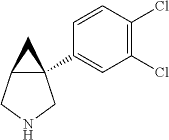 [SMILES: Clc1ccc([C@]23CNC[C@H]2C3)cc1Cl]

### (+)-1-(3,4-dichlorophenyl)-3-azabicyclo[3.1.0]hexane

(+)-1-(3,4-dichlorophenyl)-3-azabicyclo[3.1.0]hexane agents as used herein are substantially free of the corresponding (−) enantiomer, (−)-1-(3,4-dichlorophenyl)-3-azabicyclo[3.1.0]hexane. The term “substantially free of its corresponding (−)-enantiomer” means containing no more than about 5% w/w of the corresponding (−)-enantiomer, preferably no more than about 2% w/w of the corresponding (−)-enantiomer, more preferably no more than about 1% w/w of the corresponding (−)-enantiomer.

The term “corresponding (−)-enantiomer” when used in connection with (+)-1-(3,4-dichlorophenyl)-3-azabicyclo[3.1.0]hexane or a pharmaceutically acceptable salt thereof means “(−)-1-(3,4 dichlorophenyl)-3-azabicyclo[3.1.0]hexane” or a pharmaceutically acceptable salt thereof .

Synthesis of (+)-1-(3,4-dichlorophenyl)-3-azabicyclo[3.1.0]hexane may be carried out by methods described in U.S. Provisional Patent Application No. 61/419,769.

While (+)-1-(3,4-dichlorophenyl)-3-azabicyclo[3.1.0]hexane is a preferred triple reuptake inhibitor for use in the methods of the present invention, other triple reuptake inhibitors, particularly those that are unbalanced triple reuptake inhibitors, may be used as well. In a preferred embodiment, an unbalanced triple reuptake inhibitor is used that preferentially inhibits the reuptake of serotonin and norepinephrine over dopamine. For example, a triple reuptake inhibitor may have an unbalanced serotonin-norepinephrine-dopamine reuptake inhibition ratio ranging from ˜1-2:1-3:4-12, respectively. Exemplary triple reuptake inhibitor compounds include, but are not limited to, those described in U.S. patent application Ser. Nos. 12/334,432 and 12/895,788, each of which is incorporated herein by reference.

The methods of the present invention utilize (+)-1-(3,4-dichlorophenyl)-3-azabicyclo[3.1.0]hexane as well as other unbalanced triple reuptake inhibitors. It is noteworthy that others have sought to use triple reuptake inhibitors with the prospect of treating a range of central nervous disorders associated with monoamine neurotransmitters. However, in several instances, triple reuptake inhibitors have been used with little to no success in several instances due to critical shortcomings. The triple reuptake inhibitor PRC200-SS was examined as a potential antidepressant. However, further development and use of this compound is unlikely due to nephrotoxicity, as it was reported to cause acute degenerative damage of kidney tubules in a trial with cynomolgus monkeys (Guha et al., 2011). Tensofensine is a triple reuptake inhibitor that has been tested in Phase II clinical trials. The compound was reported to not be efficacious in a trial for treatment of Parkinson's Disease (Rascol et al., 2008), and was reported to be associated with gastrointestinal adverse events (dry mouth, nausea, abdominal pain, constipation, diarrhea) in a trial for its use in weight loss (Astrup et al., 2008). The triple inhibitor compound SEP-225289 underwent Phase II clinical testing for use as an antidepressant, and did not meet the primary efficacy endpoint compared to placebo (Sepracor Press Release, Jul. 1, 2009). SEP-225289 was subsequently shown by positron emission tomography occupancy study to achieve serotonin transporter occupancies that were too low at the doses examined to produce an antidepressant effect (DeLorenzo et al., 2011). Likewise, NS2359 (or GSK372475), a balanced triple reuptake inhibitor targeted for use as an antidepressant, also failed to demonstrate a significant benefit in comparison to placebo. (Neurosearch Press release, Feb. 4, 2009; Wilens et at. 2008). The triple inhibitor sibutramine was voluntarily withdrawn from the market as a weight loss/obesity drug after it was demonstrated in a clinical trial to cause a significant increase in the risk of serious heart events, including non-fatal heart attack and non-fatal stroke, in patients with a preexisting carviovascular condition (FDA News Release, 2010; James et al., 2010).

A “patient” is an animal, including, but not limited to, an animal such a cow, monkey, horse, sheep, pig, chicken, turkey, quail, cat, dog, mouse, rat, rabbit, and guinea pig, and is more preferably a mammal, and most preferably a human.

The phrase “pharmaceutically acceptable salt” as used herein is a salt formed from an acid and the basic nitrogen group of (+)-1-(3,4-dichlorophenyl)-3-azabicyclo[3.1.0]hexane. Exemplary salts include, but are not limited, to sulfate, citrate, acetate, oxalate, chloride, bromide, iodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1, I-methylenebis-(2-hydroxy-3-naphthoate)) salts.

In accordance with the invention, (+)-1-(3,4-dichlorophenyl)-3-azabicyclo[3.1.0]hexane or a pharmaceutically acceptable salt thereof is administered to a patient, preferably a mammal, more preferably a human, for the treatment or prevention of a disorder alleviated by inhibiting dopamine reuptake. In one embodiment“treatment” or “treating” refers to an amelioration of a disorder alleviated by inhibiting dopamine reuptake, or at least one discernible symptom thereof. In another embodiment, “treatment” or “treating” refers to an amelioration of at least one measurable physical parameter, not necessarily discernible by the patient. In yet another embodiment, “treatment” or “treating” refers to inhibiting the progression of a disorder alleviated by inhibiting serotonin, norepinephrine, and dopamine reuptake, either physically, e.g., normalization of a discernible symptom, physiologically, e.g., normalization of a physical parameter, or both. In yet another embodiment, “treatment” or “treating” refers to delaying the onset of a disorder alleviated by inhibiting serotonin, norepinephrine, and dopamine reuptake.

In certain embodiments, (+)-1-(3,4-dichlorophenyl)-3-azabicyclo[3.1.0]hexane or a pharmaceutically acceptable salt thereof is administered to a patient, preferably a mammal, more preferably a human, as a preventative measure against acquiring an addictive disorder. As used herein, “prevention” or “preventing” refers to a reduction of the risk of acquiring a disorder or to the reduction of the risk of recurrence of the disorder once cured or restored to a normal state. In one embodiment, (+)-1-(3,4-dichlorophenyl)-3-azabicyclo[3.1.0]hexane or a pharmaceutically acceptable salt thereof is administered as a preventative measure to a patient. According to this embodiment, the patient can have a genetic predisposition to an addictive disorder alleviated by inhibiting dopamine reuptake, such as a family history of such a disorder, or a non-genetic predisposition to an addictive disorder.

A method of the present invention is useful for treating or preventing endogenous disorders alleviated by inhibiting serotonin, norepinephrine, and dopamine uptake. Such disorders include, but are not limited to, alcohol-related, addictive and substance abuse disorders.

Disorders alleviated by inhibiting serotonin, norepinephrine, and dopamine uptake are not limited to the specific disorders described herein, and the methods of the invention will be understood or readily ascertained to provide effective treatment agents for treating and/or preventing a wide range of additional disorders and associated symptoms. For example, the methods of the invention will provide promising candidates for treatment and/or prevention of forms and symptoms of alcohol abuse, drug abuse, cognitive disorders, gambling addiction, smoking, and abnormal sexual behaviors. Additional disorders contemplated for treatment employing the methods of the invention are described, for example, in the Quick Reference to the Diagnostic Criteria from DSM-IV (Diagnostic and Statistical Manual of Mental Disorders, Fourth Edition), The American Psychiatric Association, Washington, D.C., 1994. Specific disorders whose definitions can be found in this reference are described below.

Addictive disorders amenable for treatment and/or prevention employing the methods and compositions of the invention include, but are not limited to, eating disorders, impulse control disorders, alcohol-related disorders, nicotine-related disorders, amphetamine-related disorders, cannabis-related disorders, cocaine-related disorders, hallucinogen use disorders, inhalant-related disorders, and opioid-related disorders, all of which are further sub-classified as listed below.

Alcohol-related disorders include, but are not limited to, Alcohol-Induced Psychotic Disorder, with delusions; Alcohol Abuse; Alcohol Intoxication; Alcohol Withdrawal; Alcohol Intoxication Delirium; Alcohol Withdrawal Delirium; Alcohol-Induced Persisting Dementia; Alcohol-Induced Persisting Amnestic Disorder; Alcohol Dependence; Alcohol-Induced Psychotic Disorder, with hallucinations; Alcohol-Induced Mood Disorder; Alcohol-Induced Anxiety Disorder; Alcohol-Induced Sexual Dysfunction; Alcohol-Induced Sleep Disorders; Alcohol-Related Disorders not otherwise specified (NOS); Alcohol Intoxication; and Alcohol Withdrawal.

With respect to alcohol-related disorders, including but not limited to Alcohol Abuse and Alcohol Dependence, (+)-1-(3,4-dichlorophenyl)-3-azabicyclo[3.1.0]hexane and pharmaceutically acceptable salts thereof can be used to decrease ethanol consumption associated with such alcohol-related disorders. Accordingly, the present invention provides a method for treating or preventing ethanol consumption, comprising administering to a patient in need of such treatment or prevention an effective amount (+)-1-(3,4-dichlorophenyl)-3-azabicyclo[3.1.0]hexane or a pharmaceutically acceptable salt thereof. The present invention also provides a method for treating or preventing ethanol consumption and depression, comprising administering to a patient in need of such treatment or prevention an effective amount of (+)-1-(3,4-dichlorophenyl)-3-azabicyclo[3.1.0]hexane or a pharmaceutically acceptable salt thereof. The present invention further provides pharmaceutical compositions for treating or preventing ethanol consumption in a patient comprising an effective amount of (+)-1-(3,4-dichlorophenyl)-3-azabicyclo[3.1.0]hexane or a pharmaceutically acceptable salt thereof. The present invention also provides pharmaceutical compositions for treating or preventing ethanol consumption and depression in a patient comprising (+)-1-(3,4-dichlorophenyl)-3-azabicyclo[3.1.0]hexane or a pharmaceutically acceptable salt thereof.

The routes of administration, dosage amounts and dosage forms described herein can be utilized for the administration of (+)-1-(3,4-dichlorophenyl)-3-azabicyclo[3.1.0]hexane or a pharmaceutically acceptable salt thereof for the prevention or treatment of ethanol consumption or both ethanol consumption and depression to a patient in need of such treatment. Suitable forms of the (+)-1-(3,4-dichlorophenyl)-3-azabicyclo[3.1.0]hexane for use in biologically active compositions and methods of the present invention include its pharmaceutically acceptable salts, polymorphs, solvates, hydrates, and prodrugs.

Administration of an effective amount of (+)-1-(3,4-dichlorophenyl)-3-azabicyclo[3.1.0]hexane azabicyclo[3.1.0]hexane or a pharmaceutically acceptable salt thereof: whether alone or in combination with a secondary therapeutic agent, to a patient will detectably treat or prevent ethanol consumption or both ethanol consumption and depression in the patient. In exemplary embodiments, administration of a (+)-1-(3,4-dichlorophenyl)-3-azabicyclo[3.1.0]hexane azabicyclo[3.1.0]hexane or a pharmaceutically acceptable salt thereof, whether alone or in combination with a secondary therapeutic agent, to a patient will yield a reduction in ethanol consumption, both ethanol consumption and depression, or alcohol withdrawal symptoms by at least 0%, 20%, 30%, 50% or greater, up to a 75-90%, or 95% or greater, reduction in ethanol consumption or both ethanol consumption and depression.

Nicotine-related disorders include, but are not limited to, Nicotine Dependence, Nicotine Withdrawal, and Nicotine-Related Disorder not otherwise specified (NOS).

Amphetamine-related disorders include, but are not limited to, Amphetamine Dependence, Amphetamine Abuse, Amphetamine Intoxication, Amphetamine Withdrawal, Amphetamine Intoxication Delirium, Amphetamine-Induced Psychotic Disorder with delusions, Amphetamine-Induced Psychotic Disorders with hallucinations, Amphetamine-Induced Mood Disorder, Amphetamine-Induced Anxiety Disorder, Amphetamine-Induced Sexual Dysfunction, Amphetamine-Induced Sleep Disorder, Amphetamine Related Disorder not otherwise specified (NOS), Amphetamine Intoxication, and Amphetamine Withdrawal.

Cannabis-related disorders include, but are not limited to, Cannabis Dependence; Cannabis Abuse; Cannabis Intoxication; Cannabis Intoxication Delirium; Cannabis-Induced Psychotic Disorder, with delusions; Cannabis-Induced Psychotic Disorder with hallucinations; Cannabis-Induced Anxiety Disorder; Cannabis Related Disorder not otherwise specified (NOS); and Cannabis Intoxication.

Cocaine-related disorders include, but are not limited to, Cocaine Dependence, Cocaine Abuse, Cocaine Intoxication, Cocaine Withdrawal, Cocaine Intoxication Delirium, Cocaine-Induced Psychotic Disorder with delusions, Cocaine-Induced Psychotic Disorders with hallucinations, Cocaine-Induced Mood Disorder, Cocaine-Induced Anxiety Disorder, Cocaine-Induced Sexual Dysfunction, Cocaine-Induced Sleep Disorder, Cocaine Related Disorder not otherwise specified (NOS), Cocaine Intoxication, and Cocaine Withdrawal.

Hallucinogen-use disorders include, but are not limited to, Hallucinogen Dependence, Hallucinogen Abuse, Hallucinogen Intoxication, Hallucinogen Withdrawal, Hallucinogen Intoxication Delirium, Hallucinogen-Induced Psychotic Disorder with delusions, Hallucinogen-Induced Psychotic Disorders with hallucinations, Hallucinogen-Induced Mood Disorder, Hallucinogen-Induced Anxiety Disorder, Hallucinogen-Induced Sexual Dysfunction, Hallucinogen-Induced Sleep Disorder, Hallucinogen Related Disorder not otherwise specified (NOS), Hallucinogen Intoxication, and Hallucinogen Persisting Perception Disorder (Flashbacks).

Inhalant-related disorders include, but are not limited to, Inhalant Dependence; Inhalant Abuse; Inhalant Intoxication; Inhalant Intoxication Delirium; Inhalant-Induced Psychotic Disorder, with delusions; Inhalant-Induced Psychotic Disorder with hallucinations; Inhalant-Induced Anxiety Disorder; Inhalant Related Disorder not otherwise specified (NOS); and Inhalant Intoxication.

Opioid-related disorders include, but are not limited to, Opioid Dependence, Opioid Abuse, Opioid Intoxication, Opioid Intoxication Delirium, Opioid-Induced Psychotic Disorder with delusions, Opioid-Induced Psychotic Disorder with hallucinations, Opioid-Induced Anxiety Disorder, Opioid Related Disorder not otherwise specified (NOS), Opioid Intoxication, and Opioid Withdrawal.

In certain embodiments of the present invention, (+)-1-(3,4-dichlorophenyl)-3-azabicyclo[3.1.0]hexane or a pharmaceutically acceptable salt thereof can be used in combination therapy with at least one other therapeutic agent. (+)-1-(3,4-dichlorophenyl)-3-azabicyclo[3.1.0]hexane or a pharmaceutically acceptable salt thereof and the other therapeutic agent can act additively or, more preferably, synergistically. In a preferred embodiment, (+)-1-(3,4-dichlorophenyl)-3-azabicyclo[3.1.0]hexane or a pharmaceutically acceptable salt thereof is administered concurrently with the administration of another therapeutic agent, which can be part of the same composition as or in a different composition from that comprising (+)-1-(3,4-dichlorophenyl)-3-azabicyclo[3.1.0]hexane or a pharmaceutically acceptable salt thereof. The other therapeutic agent can be useful for treating and/or preventing (as defined herein) a secondary malady resulting from a disorder alleviated by inhibiting dopamine reuptake. In another embodiment, (+)-1-(3,4 dichlorophenyl)-3-azabicyclo[3.1.0]hexane or a pharmaceutically acceptable salt thereof is administered prior or subsequent to administration of another therapeutic agent. As many of the disorders for which (+)-1-(3,4-dichlorophenyl)-3-azabicyclo[3.1.0]hexane and pharmaceutically acceptable salts thereof are useful in treating are chronic, in one embodiment combination therapy involves alternating between administering a composition comprising (+)-1-(3,4-dichlorophenyl)-3-azabicyclo[3.1.0]hexane or a pharmaceutically acceptable salt thereof and a composition comprising another therapeutic agent. The duration of administration of (+)-1-(3,4-dichlorophenyl)-3-azabicyclo[3.1.0]hexane, a pharmaceutically acceptable salt thereof, or the other therapeutic agent can be, e.g., one month, three months, six months, a year, or for more extended periods, such as the patient's lifetime. In certain embodiments, when (+)-1-(3,4-dichlorophenyl)-3-azabicyclo[3.1.0]hexane or a pharmaceutically acceptable salt thereof is administered concurrently with another therapeutic agent that potentially produces adverse side effects including, but not limited to, toxicity, the other therapeutic agent can advantageously be administered at a dose that falls below the threshold at which the adverse side effect is elicited.

The present invention also includes combinatorial formulations and coordinate administration methods which employ an effective amount of (+)-1-(3,4-dichlorophenyl)-3-azabicyclo[3.1.0]hexane (or a pharmaceutically effective salt, solvate, hydrate, polymorph, or prodrug thereof), and one or more additional active agent(s) that is/are combinatorially formulated or coordinately administered with (+)-1-(3,4-dichlorophenyl)-3-azabicyclo[3.1.0]hexane to yield a combinatorial formulation or coordinate administration method that is effective to prevent or treat ethanol consumption or both ethanol consumption and depression in a patient. Exemplary combinatorial formulations and coordinate treatment methods in this context include, for example, an effective amount of (+)-1-(3,4-dichlorophenyl)-3-azabicyclo[3.1.0]hexane in combination with one or more additional or adjunctive treatment agents or methods for preventing or treating ethanol consumption or ethanol consumption, alcohol withdrawal symptoms, and depression in a patient, such as one or more anti-alcohol or anti-depressant agent(s) and/or therapeutic method(s).

In related embodiments of the invention, (+)-1-(3,4-dichlorophenyl)-3-azabicyclo[3.1.0]hexane (or a pharmaceutically effective salt, solvate, hydrate, polymorph, or prodrug thereof) can be used in combination therapy with at least one other therapeutic agent or method. In this context, (+)-1-(3,4-dichlorophenyl)-3-azabicyclo[3.1.0]hexane can be administered concurrently or sequentially with administration of a second therapeutic agent, for example a second agent that acts to treat or prevent ethanol consumption or both ethanol consumption and depression or prevent or treat a different disorder or symptom(s) from which (+)-1-(3,4-dichlorophenyl)-3-azabicyclo[3.1.0]hexane is administered. The (+)-1-(3,4-dichlorophenyl)-3-azabicyclo[3.1.0]hexane and the second therapeutic agent can be combined in a single composition or administered in different compositions. The coordinate administration may be done simultaneously or sequentially in either order, and there may be a time period while only one or both (or all) active therapeutic agents, individually and/or collectively, exert their biological activities and therapeutic effects. A distinguishing aspect of all such coordinate treatment methods is that the (+)-1-(3,4-dichlorophenyl)-3-azabicyclo[3.1.0]hexane exerts at least some detectable therapeutic activity towards treating or preventing ethanol consumption or both ethanol consumption and depression, which may or may not be in conjunction with a secondary clinical response provided by the secondary therapeutic agent. Often, the coordinate administration of (+)-1-(3,4-dichlorophenyl)-3-azabicyclo[3.1.0]hexane with a secondary therapeutic agent as contemplated herein will yield an enhanced therapeutic response beyond the therapeutic response elicited by either or both (+)-1-(3,4-dichlorophenyl)-3-azabicyclo[3.1.0]hexane and/or secondary therapeutic agent alone.

Since (+)-1-(3,4-dichlorophenyl)-3-azabicyclo[3.1.0]hexane may need to be administered to a patient chronically for the purpose of preventing or treating ethanol consumption or both ethanol consumption and depression, in one embodiment combination therapy involves alternating between administering (+)-1-(3,4-dichlorophenyl)-3-azabicyclo[3.1.0]hexane (or a pharmaceutically effective salt, solvate, hydrate, polymorph, or prodrug thereof) and a second therapeutic agent (i.e., alternating therapy regimens between the two drugs, e.g., at one week, one month, three month, six month, or one year intervals). Alternating drug regimens in this context will often reduce or even eliminate adverse side effects, such as toxicity, that may attend long-term administration of one or both drugs alone

The other therapeutic agent can be an anti-addictive-disorder agent. Useful anti-addictive-disorder agents include, but are not limited to, tricyclic antidepressants; MAO inhibitors; glutamate agonists and antagonists, such as ketamine HCl, dextromethorphan, dextrorphan tartrate and dizocilpine (MK801); degrading enzymes, such as anesthetics and aspartate antagonists; GABA agonists, such as baclofen and muscimol HBr; reuptake blockers; degrading enzyme blockers; glutamate agonists, such as D-cycloserine, carboxyphenylglycine, L-glutamic acid, and cis-piperidinc-2,3-dicarboxylic acid; aspartate agonists; GABA antagonists such as gabazine (SR-95531), saclofen, bicuculline, picrotoxin, and (+) apomorphine HCl; and dopamine antagonists, such as spiperone HCl, haloperidol, and (−) sulpiride.

The other therapeutic agent can be an anti-alcohol agent. Useful anti-alcohol agents include, but are not limited to: disulfiram (Litten R Z et al., Development of medications for alcohol use disorders: recent advances and ongoing challenges, Expert Opin Emerg Drugs 10(2):323-43, 2005); naltrexone (Volpicelli, J. R. et al., Naltrexone in the treatment of alcohol dependence, Arch Gen Psychiatry 49:876-880, 1992; O'Malley, S. S. et al., Naltrexone and coping skills therapy for alcohol dependence: A controlled study, Arch Gen Psychiatry 49(11):881-887, 1992); acamprosate (Campral) (Swift, R. M. Drug therapy for alcohol dependence, N Engl J Med 340(19):1482-1490, 1999); ondansetron (Zofran®)(Pettinati, H. M. et al., Sertraline treatment for alcohol dependence: Interactive effects of medication and alcohol subtype, Alcohol Clin Exp Res 24(7):1041-1049, 2000; Stoltenberg, Scott F.; Serotonergic Agents and Alcoholism Treatment: A Simulation, Alcoholism: Clinical & Experimental Research 27(12):1853-1859, 2003); sertraline (Zoloft®) (Pettinati, H. M et al., Sertraline treatment for alcohol dependence: Interactive effects of medication and alcohol subtype, Alcohol Clin Exp Res 24(7):1041-1049, 2000); tiapride (Shaw G K et al., Tiapride in the long-term management of alcoholics of anxious or depressive temperament, Br J Psychiatry 150:164-8, 1987); gamma hydroxybutrate (Alcover) (Poldrugo F. and Addolorato G., The role of g-hydroxybutyric acid in the treatment of alcoholism: from animal to clinical studies. Alcohol Alcoholism 34(1), 15-24, 1999); galanthamine (Novel pharmacotherapies and patents for alcohol abuse and alcoholism 1998-2001, Expert Opinion on Therapeutic Patents, Vol. 11, No. 10, pages 1497-1521 (2001); U.S. Pat. No. 5,932,238); nalmefene (Revex) (Drobes D J et al., Effects of naltrexone and nalmefene on subjective response to alcohol among non-treatment-seeking alcoholics and social drinkers, Alcohol Clin Exp Res., 28(9):1362-70 (2004); naloxone (Julius, D., and Renault, P., eds., Narcotic Antagonists: Naltrexone Progress Report, NIDA Research Monograph Series, Number 9. DHEW Publication No. (ADM) 76-387, Bethesda, Md. National Institute on Drug Abuse, 1976; Jenab, S., and Inturrisi, C. E., Ethanol and naloxone differentially upregulate delta opioid receptor gene expression in neuroblastoma hybrid (NG108-15) cells, Molecular Brain Research 27:95-102, 1994); desoxypeganine (Doetkotte, R A et al., Effects Of Single And Repeated Administered Desoxypeganine On Ethanol Consumption Of Aa-Rats, Alcoholism: Clinical & Experimental Research, International Society for Biomedical Research on Alcoholism 12th World Congress on Biomedical Alcohol Research, Sep. 29-Oct. 2, 2004, Heidelburg/Mannheim, Germany, 28(8) Supplement:25A, 2004); benzodiazepines (Ntais C et al., Benzodiazepines for alcohol withdrawal, Cochrane Database Syst Rev. (3):CD005063, 2005; Mueller T I et al., Long-term use of benzodiazepines in participants with comorbid anxiety and alcohol use disorders, Alcohol Clin Exp Res. 29(8):1411-8, 2005); neuroleptics such as laevomepromazine (Neurocil®) and thioridazine (Melleril.®); piracetam; clonidine; carbamazepine; clomethiazole (Distraneurin®); levetiracetam; quetiapine (Monnelly E P et al., Quetiapine for treatment of alcohol dependence, J Clin Psychopharmacol. 24(5):532-5, 2004); risperidone; rimonabant; trazodone (Janiri L et al., Adjuvant trazodone in the treatment of alcoholism: an open study, Alcohol 33(4):362-5, 1998); topiramate (Johnson B A et al., Oral topiramate for treatment of alcohol dependence: a randomized controlled trial, Lancet 361:1677-1685, 2003); aripiprazole (Beresford T P et al., Aripiprazole in schizophrenia with cocaine dependence: a pilot study. J Clin Psychopharmacol. 25(4):363-6, 2005); and modafinil (Saletu B et al., On the treatment of the alcoholic organic brain syndrome with an alpha-adrenergic agonist modafinil: double-blind, placebo-controlled clinical, psychometric and neurophysiological studies, Prog Neuropsychopharmacol Biol Psychiatry 14(2):195-214, 1990).

The other therapeutic agent can be an anti-nicotine agent. Useful anti-nicotine agents include, but are not limited to, clonidine and bupropion.

The other therapeutic agent can be an anti-opiate agent. Useful anti-opiate agents include, but are not limited to, methadone, clonidine, lofexidine, levomethadyl acetate HCI, naltrexone, and buprenorphine.

The other therapeutic agent can be an anti-cocaine agent. Useful anti-cocaine agents include, but are not limited to, desipramine, amantadine, fluoxidine, d-amphetamine and buprenorphine.

The other therapeutic agent can be an appetite suppressant. Useful appetite suppressants include, but are not limited to, fenfluramine, phenylpropanolamine, and mazindol.

The other therapeutic agent can be an anti-lysergic acid diethylamide (“anti-LSD”) agent. Useful anti-LSD agents include, but are not limited to, diazepam.

The other therapeutic agent can be an anti-phencyclidine (“anti-PCP”) agent. Useful anti-PCP agents include, but are not limited to, haloperidol.

The other therapeutic agent can be an anti-Parkinson's-disease agent. Useful anti-Parkinson's-disease agents include, but are not limited to, dopamine precursors, such as levodopa, L-phenylalanine, and L-tyrosine; neuroprotective agents; dopamine agonists; dopamine reuptake inhibitors; anticholinergics such as amantadine and memantine; and 1,3,5-trisubstituted adamantanes, such as 1-amino-3,5-dimethyl-adamantane (U.S. Pat. No. 4,122,193 to Sherm et al.).

The other therapeutic agent can be an anti-depression agent. Useful anti-depression agents include, but are not limited to, amitriptyline, clomipramine, doxepine, duloxetine, imipramine, trimipramine, amoxapine, desipramine, maprotiline, nortriptyline, protripylinc, fluoxetine, fluvoxamine, mirtazepine, paroxetine, sertraline, venlafaxine, bupropion, nefazodone, trazodone, phenelzine, tranylcypromine, selegiline, clonidine, gabapentin, bicifadine and 2-pyridinyl[7-(pyridine-4-yl)pyrazolo[1,5-a]pyrimidin-3-yl]methanone compounds having at least one substituent on both the 2- and 4-pyridinyl rings. Useful classes of antidepressant agents include without limitation monoamine oxidase inhibitors, selective serotonin reuptake inhibitors, tricyclic antidepressants, tetracyclic antidepressants, norepinephrine uptake inhibitors, selective norepinephrine reuptake inhibitors, and serotonin and norepinephrine uptake inhibitors.

The other therapeutic agent can be an anxiolytic agent. Useful anxiolytic agents include, but are not limited to, benzodiazepines, such as alprazolam, chlordiazepoxide, clonazepam, clorazepate, diazepam, halazepam, lorazepam, oxazepam, and prazepam; non-benzodiazepine agents, such as buspirone; and tranquilizers, such as barbituates.

The other therapeutic agent can be an anxiolytic agent. Useful anxiolytic agents include, but are not limited to, benzodiazepines, such as alprazolam, chlordiazepoxide, clonazepam, clorazepate, diazepam, halazepam, lorazepam, oxazepam, and prazepam; non-benzodiazepine agents, such as buspirone; and tranquilizers, such as barbituates.

The other therapeutic agent can be an antipsychotic drug. Useful antipsychotic drugs include, but are not limited to, phenothiazines, such as chlorpromazine, mesoridazine besylate, thioridazine, acetophenazine maleate, fluphenazine, perphenazine, and trifluoperazine; thioxanthenes, such as chlorprothixene, and thiothixene; and other heterocyclic compounds, such as clozapine, haloperidol, loxapine, molindonc, pimozide, and risperidone. Additional antipsychotic drugs include olanzapine, aripiprazole, quetiapine, and ziprasidone. Preferable anti-psychotic drugs include chlorpromazine HCI, thioridazine HCl, fluphenazine HCI, thiothixene HCI, and molindone HCI.

The other therapeutic agent can be an anti-obesity drug. Useful anti-obesity drugs include, but are not limited, to β-adrenergic receptor agonists, preferably β-3 receptor agonists such as, but not limited to, fenfluramine; dexfenfluramine; sibutramine; bupropion; fluoxetine; phentermine; amphetamine; methamphetamine; dextroamphetamine; benzphetamine; phendimetrazine; diethylpropion; mazindol; phenylpropanolamine; norepinephrine; serotonin reuptake inhibitors, such as sibutramine; and pancreatic lipase inhibitors, such as orlistat.

Administration of an effective amount (+)-1-(3,4-dichlorophenyl)-3-azabicyclo[3.1.0]hexane in the methods described herein to a mammalian subject presenting with one or more symptoms of an alcohol-related or addictive disorder or other neurological or psychiatric condition will detectably decrease, eliminate, or prevent the targeted disorder and/or associated symptom(s). In exemplary embodiments, administration of a (+)-1-(3,4-dichlorophenyl)-3-azabicyclo[3.1.0]hexane composition to a suitable test subject will yield a reduction in one or more target symptom(s) associated with a selected disorder, such as pain, by at least 10%, 20%, 30%, 50% or greater, up to a 75-90%, or 95% or greater, reduction in the targeted disorder or one or more target symptom(s), compared to placebo-treated or other suitable control subjects. Comparable levels of efficacy are contemplated for the entire range of alcohol-related or addictive disorders, and related conditions and symptoms, for treatment or prevention using the methods of the invention.

An “effective amount,” “therapeutic amount,” “therapeutically effective amount,” or “effective dose” of (+)-1-(3,4-dichlorophenyl)-3-azabicyclo[3.1.0]hexane agent and/or a psychotherapeutic agent as used herein means an effective amount or dose of the active compound as described herein sufficient to elicit a desired pharmacological or therapeutic effect in a human subject. Such an effect typically results in a measurable reduction in an occurrence, frequency, or severity of one or more symptom(s) of a disorder, including any combination of neurological and/or psychological symptoms, diseases, or conditions, associated with or caused by the targeted disorder, in the subject. In certain embodiments, when a compound as described herein is administered to treat a disorder, an effective amount of the compound will be an amount sufficient in vivo to delay or eliminate onset of symptoms, or delay or eliminate harmful activities of the targeted condition or disorder.

Therapeutic efficacy can alternatively be demonstrated by a decrease in the frequency or severity of symptoms associated with the treated alcohol-related or addictive condition or disorder, or by altering the nature, occurrence, recurrence, or duration of symptoms associated with the treated condition or disorder. In this context, “effective amounts,” “therapeutic amounts,” “therapeutically effective amounts,” and “effective doses” of triple reuptake inhibitor agents described herein can be readily determined by ordinarily skilled artisans following the teachings of this disclosure and employing tools and methods generally known in the art, often based on routine clinical or patient-specific factors.

Suitable routes of administration for a (+)-1-(3,4-dichlorophenyl)-3-azabicyclo[3.1.0]hexane agent in the methods disclosed herein include, but are not limited to, oral, buccal, nasal, aerosol, topical, transdermal, mucosal, injectable, slow release, controlled release, iontophoresis, sonophoresis, and other conventional delivery routes, devices and methods. Injectable delivery methods are also contemplated, including but not limited to, intravenous, intramuscular, intraperitoneal, intraspinal, intrathecal, intracerebroventricular, intraarterial, and subcutaneous injection.

Suitable effective unit dosage amounts of a (+)-1-(3,4-dichlorophenyl)-3-azabicyclo[3.1.0]hexane compound used as disclosed herein for mammalian subjects may range from about 1 to about 1800 mg, about 10 to about 1800 mg, 25 to about 1800 mg, about 50 to about 1000 mg, about 75 to about 900 mg, about 100 to about 750 mg, or about 150 to about 500 mg. In certain embodiments, the effective dosage will be selected within narrower ranges of, for example, about 5 to about 10 mg, 10 to about 25 mg, about 30 to about 50 mg, about 10 to about 300 mg, about 25 to about 300 mg, about 50 to about 100 mg, about 100 to about 250 mg, or about 250 to about 500 mg. These and other effective unit dosage amounts may be administered in a single dose, or in the form of multiple daily, weekly or monthly doses, for example in a dosing regimen comprising from 1 to 4, or 2-3, doses administered per day, per week, or per month. In exemplary embodiments, dosages of about 10 to about 25 mg, about 30 to about 50 mg, about 25 to about 150, about 50 to about 100 mg, about 100 to about 250 mg, or about 250 to about 500 mg, are administered one, two, three, or four times per day. In more detailed embodiments, dosages of about 50-75 mg, about 100-200 mg, about 250-400 mg, or about 400-600 mg are administered once or twice daily. In further detailed embodiments, dosages of about 50-100 mg are administered twice daily. In alternate embodiments, dosages are calculated based on body weight, and may be administered, for example, in amounts from about 0.5 mg/kg to about 20 mg/kg per day, 1 mg/kg to about 15 mg/kg per day, 1 mg/kg to about 10 mg/kg per day, 2 mg/kg to about 20 mg/kg per day, 2 mg/kg to about 10 mg/kg per day or 3 mg/kg to about 15 mg/kg per day

The amount, timing, and mode of delivery of compositions comprising an effective amount of a (+)-1-(3,4-dichlorophenyl)-3-azabicyclo[3.1.0]hexane agent as used in the methods described herein will be routinely adjusted on an individual basis, depending on such factors as weight, age, gender, and condition of the individual, the acuteness of the condition to be treated and/or related symptoms, whether the administration is prophylactic or therapeutic, and on the basis of other factors known to effect drug delivery, absorption, pharmacokinetics, including half-life, and efficacy. An effective dose or multi-dose treatment regimen for the compounds of the invention will ordinarily be selected to approximate a minimal dosing regimen that is necessary and sufficient to substantially prevent or alleviate one or more symptom(s) of a neurological or psychiatric condition in the subject, as described herein. Thus, following administration of a a (+)-1-(3,4-dichlorophenyl)-3-azabicyclo[3.1.0]hexane compound or pharmaceutically acceptable salt thereof according to the formulations and methods herein, test subjects will exhibit a 10%, 20%, 30%, 50% or greater reduction, up to a 75-90%, or 95% or greater, reduction, in one or more symptoms associated with a targeted monoamine neurotransmitter influenced disorder or other neurological or psychiatric condition, compared to placebo-treated or other suitable control subjects.

Pharmaceutical dosage forms of a compound used in the present invention may optionally include excipients recognized in the art of pharmaceutical compounding as being suitable for the preparation of dosage units as discussed above. Such excipients include, without intended limitation, binders, fillers, lubricants, emulsifiers, suspending agents, sweeteners, flavorings, preservatives, buffers, wetting agents, disintegrants, effervescent agents and other conventional excipients and additives.

Pharmaceutical dosage forms of a a (+)-1-(3,4-dichlorophenyl)-3-azabicyclo[3.1.0]hexane composition may include inorganic and organic acid addition salts. The pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; organic acid salts such as acetate, citrate, lactate, succinate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts such as arginate, asparginate, glutamate, tartrate, gluconate and the like.

Within various combinatorial or coordinate treatment methods disclosed herein, the additional therapeutic agent and a (+)-1-(3,4-dichlorophenyl)-3-azabicyclo[3.1.0]hexane compound or a pharmaceutically acceptable salt thereof may each be administered by any of a variety of delivery routes and modes, which may be the same or different for each agent.

An additional psychotherapeutic compound and/or a (+)-1-(3,4-dichlorophenyl)-3-azabicyclo[3.1.0]hexane administered according to the present invention will often be formulated and administered in an oral dosage form, optionally in combination with a carrier or other additive(s). Suitable carriers common to pharmaceutical formulation technology include, but are not limited to, microcrystalline cellulose, lactose, sucrose, fructose, glucose dextrose, or other sugars, di-basic calcium phosphate, calcium sulfate, cellulose, methylcellulose, cellulose derivatives, kaolin, mannitol, lactitol, maltitol, xylitol, sorbitol, or other sugar alcohols, dry starch, dextrin, maltodextrin or other polysaccharides, inositol, or mixtures thereof. Exemplary unit oral dosage forms for use in this invention include tablets and capsules, which may be prepared by any conventional method of preparing pharmaceutical oral unit dosage forms can be utilized in preparing oral unit dosage forms. Oral unit dosage forms, such as tablets or capsules, may contain one or more conventional additional formulation ingredients, including, but are not limited to, release modifying agents, glidants, compression aides, disintegrants, lubricants, binders, flavors, flavor enhancers, sweeteners and/or preservatives. Suitable lubricants include stearic acid, magnesium stearate, talc, calcium stearate, hydrogenated vegetable oils, sodium benzoate, leucine carbowax, magnesium lauryl sulfate, colloidal silicon dioxide and glyceryl monostearate. Suitable glidants include colloidal silica, fumed silicon dioxide, silica, talc, fumed silica, gypsum and glyceryl monostearate. Substances which may be used for coating include hydroxypropyl cellulose, titanium oxide, talc, sweeteners and colorants. The aforementioned effervescent agents and disintegrants are useful in the formulation of rapidly disintegrating tablets known to those skilled in the art. These typically disintegrate in the mouth in less than one minute, and preferably in less than thirty seconds. By effervescent agent is meant a couple, typically an organic acid and a carbonate or bicarbonate.

A a (+)-1-(3,4-dichlorophenyl)-3-azabicyclo[3.1.0]hexane composition as disclosed herein can be prepared and administered in any of a variety of inhalation or nasal delivery forms known in the art. Devices capable of depositing aerosolized formulations of a triple reuptake inhibitor compound or a pharmaceutically acceptable salt thereof of the invention in the sinus cavity or pulmonary alveoli of a patient include metered dose inhalers, nebulizers, dry powder generators, sprayers, and the like. Pulmonary delivery to the lungs for rapid transit across the alveolar epithelium into the blood stream may be particularly useful in treating impending episodes of depression. Methods and compositions suitable for pulmonary delivery of drugs for systemic effect are well known in the art. Suitable formulations, wherein the carrier is a liquid, for administration, as for example, a nasal spray or as nasal drops, may include aqueous or oily solutions of a compound of the present invention, and any additional active or inactive ingredient(s).

Intranasal delivery permits the passage of active compounds as disclosed herein into the blood stream directly after administering an effective amount of the compound to the nose, without requiring the product to be deposited in the lung. In addition, intranasal delivery can achieve direct, or enhanced, delivery of the active compound to the central nervous system. In these and other embodiments, intranasal administration of the compounds of the invention may be advantageous for treating disorders influenced by monoamine neurotransmitters, by providing for rapid absorption and delivery.

For intranasal and pulmonary administration, a liquid aerosol formulation will often contain an active compound as described herein combined with a dispersing agent and/or a physiologically acceptable diluent. Alternative, dry powder aerosol formulations may contain a finely divided solid form of the subject compound and a dispersing agent allowing for the ready dispersal of the dry powder particles. With either liquid or dry powder aerosol formulations, the formulation must be aerosolized into small, liquid or solid particles in order to ensure that the aerosolized dose reaches the mucous membranes of the nasal passages or the lung. The term “aerosol particle” is used herein to describe a liquid or solid particle suitable of a sufficiently small particle diameter, e.g., in a range of from about 2-5 microns, for nasal or pulmonary distribution to targeted mucous or alveolar membranes. Other considerations include the construction of the delivery device, additional components in the formulation, and particle characteristics. These aspects of nasal or pulmonary administration of drugs are well known in the art, and manipulation of formulations, aerosolization means, and construction of delivery devices, is within the level of ordinary skill in the art.

Yet additional methods of the invention are provided for topical administration of a a (+)-1-(3,4-dichlorophenyl)-3-azabicyclo[3.1.0]hexane compound or a pharmaceutically acceptable salt thereof. Topical compositions may comprise a compound as described herein and any other active or inactive component(s) incorporated in a dermatological or mucosal acceptable carrier, including in the form of aerosol sprays, powders, dermal patches, sticks, granules, creams, pastes, gels, lotions, syrups, ointments, impregnated sponges, cotton applicators, or as a solution or suspension in an aqueous liquid, non-aqueous liquid, oil-in-water emulsion, or water-in-oil liquid emulsion. These topical compositions may comprise a compound as disclosed herein dissolved or dispersed in water or other solvent or liquid to be incorporated in the topical composition or delivery device. It can be readily appreciated that the transdermal route of administration may be enhanced by the use of a dermal penetration enhancer known to those skilled in the art. Formulations suitable for such dosage forms incorporate excipients commonly utilized therein, particularly means, e.g. structure or matrix, for sustaining the absorption of the drug over an extended period of time, for example 24 hours.

Yet additional formulations of a compound used in the present invention are provided for parenteral administration, including aqueous and non-aqueous sterile injection solutions which may optionally contain anti-oxidants, buffers, bacteriostats and/or solutes which render the formulation isotonic with the blood of the mammalian subject; aqueous and non-aqueous sterile suspensions which may include suspending agents and/or thickening agents; dispersions; and emulsions. The formulations may be presented in unit-dose or multi-dose containers. Pharmaceutically acceptable formulations and ingredients will typically be sterile or readily sterilizable, biologically inert, and easily administered. Parenteral preparations typically contain buffering agents and preservatives, and may be lyophilized for reconstitution at the time of administration.

Parental formulations may also include polymers for extended release following parenteral administration. Such polymeric materials are well known to those of ordinary skill in the pharmaceutical compounding arts. Extemporaneous injection solutions, emulsions and suspensions may be prepared from sterile powders, granules and tablets of the kind previously described. Preferred unit dosage formulations are those containing a daily dose or unit, daily sub-dose, as described herein above, or an appropriate fraction thereof, of the active ingredient(s).

Within exemplary compositions and dosage forms used in the methods of the invention, a a (+)-1-(3,4-dichlorophenyl)-3-azabicyclo[3.1.0]hexane compound or a pharmaceutically acceptable salt thereof for treating disorders disclosed herein is administered in an extended release or sustained release formulation. In these formulations, the sustained release composition of the formulation provides therapeutically effective plasma levels of the active compound (1R,5S)- or a pharmaceutically acceptable salt thereof over a sustained delivery period of approximately 8 hours or longer, or over a sustained delivery period of approximately 18 hours or longer, up to a sustained delivery period of approximately 24 hours or longer.

In exemplary embodiments, a (+)-1-(3,4-dichlorophenyl)-3-azabicyclo[3.1.0]hexane compound or a pharmaceutically acceptable salt thereof is combined with a sustained release vehicle, matrix, binder, or coating material. As used herein, the term “sustained release vehicle, matrix, binder, or coating material” refers to any vehicle, matrix, binder, or coating material that effectively, significantly delays dissolution of the active compound in vitro, and/or delays, modifies, or extends delivery of the active compound into the blood stream (or other in vivo target site of activity) of a subject following administration (e.g., oral administration), in comparison to dissolution and/or delivery provided by an “immediate release” formulation, as described herein, of the same dosage amount of the active compound. Accordingly, the term “sustained release vehicle, matrix, binder, or coating material” as used herein is intended to include all such vehicles, matrices, binders and coating materials known in the art as “sustained release”, “delayed release”, “slow release”, “extended release”, “controlled release”, “modified release”, and “pulsatile release” vehicles, matrices, binders and coatings.

In one aspect, the current invention comprises methods using an oral sustained release dosage composition for administering a a (+)-1-(3,4-dichlorophenyl)-3-azabicyclo[3.1.0]hexane compound or a pharmaceutically acceptable salt thereof. In a related aspect, the invention comprises a method of reducing one or more side effects that attend administration of an oral dosage form of a a (+)-1-(3,4-dichlorophenyl)-3-azabicyclo[3.1.0]hexane compound or a pharmaceutically acceptable salt thereof by employing a sustained release formulation. Within this method, following oral administration of a a (+)-1-(3,4-dichlorophenyl)-3-azabicyclo[3.1.0]hexane compound or a pharmaceutically acceptable salt thereof, the active agent is released in a sustained, delayed, gradual or modified release delivery mode into the gastrointestinal tract (e.g., the intestinal lumen) of the subject over a period of hours, during which the triple reuptake inhibitor compound or a pharmaceutically acceptable salt thereof reaches, and is sustained at, a therapeutic concentration in a blood plasma, tissue, organ or other target site of activity (e.g., a central nervous system tissue, fluid or compartment) in the patient. When following this method, the side effect profile of triple reuptake inhibitor compound or a pharmaceutically acceptable salt thereof is less than a side effect profile of an equivalent dose of a triple reuptake inhibitor compound or a pharmaceutically acceptable salt thereof administered in an immediate release oral dosage form.

In certain embodiments, a (+)-1-(3,4-dichlorophenyl)-3-azabicyclo[3.1.0]hexane compound or a pharmaceutically acceptable salt thereof is released from the sustained release compositions and dosage forms of the invention and delivered into the blood plasma or other target site of activity in the subject at a sustained therapeutic level over a period of at least about 6 hours, often over a period of at least about 8 hours, at least about 12 hours, or at least about 18 hours, and in other embodiments over a period of about 24 hours or greater. By sustained therapeutic level is meant a plasma concentration level of at least a lower end of a therapeutic dosage range as exemplified herein. In more detailed embodiments of the invention, the sustained release compositions and dosage forms will yield a therapeutic level of a triple reuptake inhibitor compound or a pharmaceutically acceptable salt thereof following administration to a mammalian subject in a desired dosage amount (e.g.,5, 10, 25, 50, 100 200, 400, 600, or 800 mg) that yields a minimum plasma concentration of at least a lower end of a therapeutic dosage range as exemplified herein over a period of at least about 6 hours, at least about 8 hours, at least about 12 hours, at least about 18 hours, or up to 24 hours or longer. In alternate embodiments of the invention, the sustained release compositions and dosage forms will yield a therapeutic level of a (+)-1-(3,4-dichlorophenyl)-3-azabicyclo[3.1.0]hexane compound or a pharmaceutically acceptable salt thereof following administration to a mammalian subject in a desired dosage amount (e.g., 5, 10, 25, 50, 100, 200, 400, 600, or 800 mg) that yields a minimum plasma concentration that is known to be associated with clinical efficacy, over a period of at least about 6 hours, at least about 8 hours, at least about 12 hours, at least about 18 hours, or up to 24 hours or longer.

In certain embodiments, a (+)-1-(3,4-dichlorophenyl)-3-azabicyclo[3.1.0]hexane compound or a pharmaceutically acceptable salt thereof is released from the compositions and dosage forms disclosed herein and delivered into the blood plasma or other target site of activity in the subject (including, but not limited to, areas of the brain such as the prefrontal cortex, frontal cortex, thalamus, striatum, ventral tegmental area, other cortical areas, hippocampus, hypothalamus, or nucleus accumbens) in a sustained release profile characterized in that from about 0% to 20% of the active compound is released and delivered (as determined, e.g., by measuring blood plasma levels) within in 0 to 2 hours, from 20% to 50% of the active compound is released and delivered within about 2 to 12 hours, from 50% to 85% of the active compound is released and delivered within about 3 to 20 hours, and greater than 75% of the active compound is released and delivered within about 5 to 18 hours.

In more detailed embodiments of the invention, compositions and oral dosage forms of a (+)-1-(3,4-dichlorophenyl)-3-azabicyclo[3.1.0]hexane compound or a pharmaceutically acceptable salt thereof are provided, wherein the compositions and dosage forms, after ingestion, provide a curve of concentration of a (+)-1-(3,4-dichlorophenyl)-3-azabicyclo[3.1.0]hexane compound or a pharmaceutically acceptable salt thereof agents over time, the curve having an area under the curve (AUC) which is approximately proportional to the dose of the (+)-1-(3,4-dichlorophenyl)-3-azabicyclo[3.1.0]hexane compound or a pharmaceutically acceptable salt thereof administered, and a maximum concentration (Cₘₐₓ) that is proportional to the dose of the (+)-1-(3,4-dichlorophenyl)-3-azabicyclo[3.1.0]hexane compound or a pharmaceutically acceptable salt thereof administered.

In other detailed embodiments, the Cₘₐₓ of a (+)-1-(3,4-dichlorophenyl)-3-azabicyclo[3.1.0]hexane compound or a pharmaceutically acceptable salt thereof provided after oral delivery of a composition or dosage form of the invention is less than about 80%, often less than about 75%, in some embodiments less than about 60%, or 50%, of a Cₘₐₓ obtained after administering an equivalent dose of the active compound in an immediate release oral dosage form.

Within exemplary embodiments of the invention, the compositions and dosage forms containing of a (+)-1-(3,4-dichlorophenyl)-3-azabicyclo[3.1.0]hexane compound or a pharmaceutically acceptable salt thereof and a sustained release vehicle, matrix, binder, or coating will yield sustained delivery of the active compound such that, following administration of the composition or dosage form to a mammalian treatment subject, the Cₘₐₓ of the (+)-1-(3,4-dichlorophenyl)-3-azabicyclo[3.1.0]hexane compound or a pharmaceutically acceptable salt thereof in the treatment subject is less than about 80% of a Cₘₐₓ provided in a control subject after administration of the same amount of the (+)-1-(3,4-dichlorophenyl)-3-azabicyclo[3.1.0]hexane compound or a pharmaceutically acceptable salt thereof in an immediate release formulation.

As used herein, the term “immediate release dosage form” refers to a dosage form of a (+)-1-(3,4-dichlorophenyl)-3-azabicyclo[3.1.0]hexane compound or a pharmaceutically acceptable salt thereof wherein the active compound readily dissolves upon contact with a liquid physiological medium, for example phosphate buffered saline (PBS) or natural or artificial gastric fluid. In certain embodiments, an immediate release formulation will be characterized in that at least 70% of the active compound will be dissolved within a half hour after the dosage form is contacted with a liquid physiological medium. In alternate embodiments, at least 80%, 85%, 90% or more, or up to 100%, of the active compound in an immediate release dosage form will dissolve within a half hour following contact of the dosage form with a liquid physiological medium in an art-accepted in vitro dissolution assay. These general characteristics of an immediate release dosage form will often relate to powdered or granulated compositions of a (+)-1-(3,4-dichlorophenyl)-3-azabicyclo[3.1.0]hexane compound or a pharmaceutically acceptable salt thereof in a capsulated dosage form, for example in a gelatin-encapsulated dosage form, where dissolution will often be relatively immediate after dissolution/failure of the gelatin capsule. In alternate embodiments, the immediate release dosage form may be provided in the form of a compressed tablet, granular preparation, powder, or even liquid dosage form, in which cases the dissolution profile will often be even more immediate (e.g., wherein at least 85%-95% of the active compound is dissolved within a half hour).

In additional embodiments of the invention, an immediate release dosage form will include compositions wherein the (+)-1-(3,4-dichlorophenyl)-3-azabicyclo[3.1.0]hexane compound or a pharmaceutically acceptable salt thereof is not admixed, bound, coated or otherwise associated with a formulation component that substantially impedes in vitro or in vivo dissolution and/or in vivo bioavailability of the active compound. Within certain embodiments, a (+)-1-(3,4-dichlorophenyl)-3-azabicyclo[3.1.0]hexane compound or a pharmaceutically acceptable salt thereof will be provided in an immediate release dosage form that does not contain significant amounts of a sustained release vehicle, matrix, binder or coating material. In this context, the term “significant amounts of a sustained release vehicle, matrix, binder or coating material” is not intended to exclude any amount of such materials, but an amount sufficient to impede in vitro or in vivo dissolution of a (+)-1-(3,4-dichlorophenyl)-3-azabicyclo[3.1.0]hexane compound or a pharmaceutically acceptable salt thereof in a formulation containing such materials by at least 5%, often at least 10%, and up to at least 15%-20% compared to dissolution of the (+)-1-(3,4-dichlorophenyl)-3-azabicyclo[3.1.0]hexane compound or a pharmaceutically acceptable salt thereof when provided in a composition that is essentially free of such materials.

In alternate embodiments of the invention, an immediate release dosage form of a (+)-1-(3,4-dichlorophenyl)-3-azabicyclo[3.1.0]hexane compound or a pharmaceutically acceptable salt thereof may be any dosage form comprising the active compound which fits the FDA Biopharmaceutics Classification System (BCS) Guidance definition (see, e.g., http://www.fda.gov/cder/OPS/BCS_guidance.htm) of a “high solubility substance in a rapidly dissolving formulation.” In exemplary embodiments, an immediate release formulation of a (+)-1-(3,4-dichlorophenyl)-3-azabicyclo[3.1.0]hexane compound or a pharmaceutically acceptable salt thereof according to this aspect of the invention will exhibit rapid dissolution characteristics according to BCS Guidance parameters, such that at least approximately 85% of (+)-1-(3,4-dichlorophenyl)-3-azabicyclo[3.1.0]hexane compound or a pharmaceutically acceptable salt thereof in the formulation will go into a test solution within about 30 minutes at pH 1, pH 4.5, and pH 6.8.

The compositions, dosage forms and methods disclosed herein thus include novel tools for coordinate treatment of disorders involving monoamine neurotransmitters by providing for sustained release and/or sustained delivery a (+)-1-(3,4-dichlorophenyl)-3-azabicyclo[3.1.0]hexane compound or a pharmaceutically acceptable salt thereof. As used herein, “sustained release” and “sustained delivery” are evinced by a sustained, delayed, extended, or modified, in vitro or in vivo dissolution rate, in vivo release and/or delivery rate, and/or in vivo pharmacokinetic value(s) or profile.

The sustained release dosage forms used in the methods of the invention can take any form as long as one or more of the dissolution, release, delivery and/or pharmacokinetic property(ies) identified above are satisfied. Within illustrative embodiments, the composition or dosage form can comprise a (+)-1-(3,4-dichlorophenyl)-3-azabicyclo[3.1.0]hexane compound or a pharmaceutically acceptable salt thereof combined with any one or combination of: a drug-releasing polymer, matrix, bead, microcapsule, or other solid drug-releasing vehicle; drug-releasing tiny timed-release pills or mini-tablets; compressed solid drug delivery vehicle; controlled release binder; multi-layer tablet or other multi-layer or multi-component dosage form; drug-releasing lipid; drug-releasing wax; and a variety of other sustained drug release materials as contemplated herein, or formulated in an osmotic dosage form.

The present invention thus encompasses a broad range of sustained release compositions and dosage forms a (+)-1-(3,4-dichlorophenyl)-3-azabicyclo[3.1.0]hexane compound or a pharmaceutically acceptable salt thereof, which in certain embodiments are adapted for providing sustained release of the active compound(s) following, e.g., oral administration. Sustained release vehicles, matrices, binders and coatings for use in accordance with the invention include any biocompatible sustained release material which is inert to the active agent and which is capable of being physically combined, admixed, or incorporated with the active compound. Useful sustained release materials may be dissolved, degraded, disintegrated, and/or metabolized slowly under physiological conditions following delivery (e.g., into a gastrointestinal tract of a subject, or following contact with gastric fluids or other bodily fluids). Useful sustained release materials are typically non-toxic and inert when contacted with fluids and tissues of mammalian subjects, and do not trigger significant adverse side effects such as irritation, immune response, inflammation, or the like. They are typically metabolized into metabolic products which are biocompatible and easily eliminated from the body.

In certain embodiments, sustained release polymeric materials are employed as the sustained release vehicle, matrix, binder, or coating (see, e.g., “Medical Applications of Controlled Release,” Langer and Wise (eds.), CRC Press., Boca Raton, Fla. (1974); “Controlled Drug Bioavailability,” Drug Product Design and Performance, Smolen and Ball (eds.), Wiley, N.Y. (1984); Ranger and Peppas, 1983, J Macromol. Sci. Rev. Macromol Chem. 23:61; see also Levy et al., 1985, Science 228: 190; During et al., 1989, Ann. Neurol. 25:351; Howard et al, 1989, J. Neurosurg. 71:105, each incorporated herein by reference). Within exemplary embodiments, useful polymers for co-formulating with a (+)-1-(3,4-dichlorophenyl)-3-azabicyclo[3.1.0]hexane compound or a pharmaceutically acceptable salt thereof to yield a sustained release composition or dosage form include, but are not limited to, ethylcellulose, hydroxyethyl cellulose; hydroxyethylmethyl cellulose; hydroxypropyl cellulose; hydroxypropylmethyl cellulose; hydroxypropylmethyl cellulose phthalate; hydroxypropylmethylcellulose acetate succinate; hydroxypropylmethylcellulose acetate phthalate; sodium carboxymethylcellulose; cellulose acetate phthalate; cellulose acetate trimellitate; polyoxyethylene stearates; polyvinyl pyrrolidone; polyvinyl alcohol; copolymers of polyvinyl pyrrolidone and polyvinyl alcohol; polymethacrylate copolymers; and mixtures thereof.

Additional polymeric materials for use as sustained release vehicles, matrices, binders, or coatings within the compositions and dosage forms used in the invention include, but are not limited to, additional cellulose ethers, e.g., as described in Alderman, Int. J. Pharm. Tech. & Prod. Mfr., 1984, 5(3) 1-9 (incorporated herein by reference). Other useful polymeric materials and matrices are derived from copolymeric and homopolymeric polyesters having hydrolysable ester linkages. A number of these are known in the art to be biodegradable and to lead to degradation products having no or low toxicity. Exemplary polymers in this context include polyglycolic acids (PGAs) and polylactic acids (PLAs), poly(DL-lactic acid-co-glycolic acid) (DL PLGA), poly(D-lactic acid-coglycolic acid) (D PLGA) and poly(L-lactic acid-co-glycolic acid) (L PLGA). Other biodegradable or bioerodable polymers for use within the invention include such polymers as poly(ε-caprolactone), poly(ε-aprolactone-CO-lactic acid), poly(ε-aprolactone-CO-glycolic acid), poly(ß-hydroxy butyric acid), poly(alkyl-2-cyanoacrilate), hydrogels such as poly(hydroxyethyl methacrylate), polyamides, poly-amino acids (e.g., poly-L-leucine, poly-glutamic acid, poly-L-aspartic acid, and the like), poly (ester ureas), poly (2-hydroxyethyl DL-aspartamide), polyacetal polymers, polyorthoesters, polycarbonates, polymaleamides, polysaccharides, and copolymers thereof. Methods for preparing pharmaceutical formulations using these polymeric materials are generally known to those skilled in the art (see, e.g., Sustained and Controlled Release Drug Delivery Systems, J. R. Robinson, ed., Marcel Dekker, Inc., New York, 1978, incorporated herein by reference).

In other embodiments of the invention, the compositions and dosage forms of a (+)-1-(3,4-dichlorophenyl)-3-azabicyclo[3.1.0]hexane compound or a pharmaceutically acceptable salt thereof are coated on a polymer substrate. The polymer can be an erodible or a nonerodible polymer. The coated substrate may be folded onto itself to provide a bilayer polymer drug dosage form. For example, a (+)-1-(3,4-dichlorophenyl)-3-azabicyclo[3.1.0]hexane compound or a pharmaceutically acceptable salt thereof can be coated onto a polymer such as a polypeptide, collagen, gelatin, polyvinyl alcohol, polyorthoester, polyacetyl, or a polyorthocarbonate, and the coated polymer folded onto itself to provide a bilaminated dosage form. In operation, the bioerodible dosage form erodes at a controlled rate to dispense the active compound over a sustained release period. Representative biodegradable polymers for use in this and other aspects of the invention can be selected from, for example, biodegradable poly(amides), poly (amino acids), poly(esters), poly(lactic acid), poly(glycolic acid), poly(carbohydrate), poly(orthoester), poly (orthocarbonate), poly(acetyl), poly(anhydrides), biodegradable poly(dehydropyrans), and poly(dioxinones) which are known in the art (see, e.g., Rosoff, Controlled Release of Drugs, Chap. 2, pp. 53-95 (1989); and U.S. Pat. Nos. 3,811,444; 3,962,414; 4,066,747, 4,070,347; 4,079,038; and 4,093,709, each incorporated herein by reference).

In another embodiment of the invention, the dosage form comprises a (+)-1-(3,4-dichlorophenyl)-3-azabicyclo[3.1.0]hexane compound or a pharmaceutically acceptable salt thereof loaded into a polymer that releases the drug by diffusion through a polymer, or by flux through pores or by rupture of a polymer matrix. The drug delivery polymeric dosage form comprises the active compound contained in or on the polymer. The dosage form comprises at least one exposed surface at the beginning of dose delivery. The non-exposed surface, when present, can be coated with a pharmaceutically acceptable material impermeable to the passage of a drug. The dosage form may be manufactured by procedures known in the art, for example by blending a pharmaceutically acceptable carrier like polyethylene glycol, with a pre-determined dose of the active compound(s) at an elevated temperature (e.g., 37° C.), and adding it to a silastic medical grade elastomer with a cross-linking agent, for example, octanoate, followed by casting in a mold. The step is repeated for each optional successive layer. The system is allowed to set for 1 hour, to provide the dosage form. Representative polymers for manufacturing such sustained release dosage forms include, but are not limited to, olefin, and vinyl polymers, addition polymers, condensation polymers, carbohydrate polymers, and silicon polymers as represented by polyethylene, polypropylene, polyvinyl acetate, polymethylacrylate, polyisobutylmethacrylate, poly alginate, polyamide and polysilicon. These polymers and procedures for manufacturing them have been described in the art (see, e.g., Coleman et al., Polymers 1990, 31, 1187-1231; Roerdink et al., Drug Carrier Systems 1989, 9, 57-10; Leong et al., Adv. Drug Delivery Rev. 1987, 1, 199-233; and Roff et al., Handbook of Common Polymers 1971, CRC Press; U.S. Pat. No. 3,992,518).

In other embodiments of the invention, the compositions and dosage forms comprise a (+)-1-(3,4-dichlorophenyl)-3-azabicyclo[3.1.0]hexane compound or a pharmaceutically acceptable salt thereof incorporated with or contained in beads that on dissolution or diffusion release the active compound over an extended period of hours, for example over a period of at least 6 hours, over a period of at least 8 hours, over a period of at least 12 hours, or over a period of up to 24 hours or longer. The drug-releasing beads may have a central composition or core comprising a (+)-1-(3,4-dichlorophenyl)-3-azabicyclo[3.1.0]hexane compound or a pharmaceutically acceptable salt thereof and a pharmaceutically acceptable carrier, along with one or more optional excipients such as a lubricants, antioxidants, dispersants, and buffers. The beads may be medical preparations with a diameter of about 1 to 2 mm. In exemplary embodiments they are formed of non-cross-linked materials to enhance their discharge from the gastrointestinal tract. The beads may be coated with a release rate-controlling polymer that gives a timed release pharmacokinetic profile. In alternate embodiments the beads may be manufactured into a tablet for therapeutically effective drug administration. The beads can be made into matrix tablets by direct compression of a plurality of beads coated with, for example, an acrylic resin and blended with excipients such as hydroxypropylmethyl cellulose. The manufacture and processing of beads for use within the invention is described in the art (see, e.g., Lu, Int. J. Pharm., 1994, 112, 117-124; Pharmaceutical Sciences by Remington, 14^{th} ed, pp1626-1628 (1970); Fincher, J. Pharm. Sci. 1968, 57, 1825-1835; and U.S. Pat. No. 4,083,949, each incorporated by reference) as has the manufacture of tablets (Pharmaceutical Sciences, by Remington, 17^{th} Ed, Ch. 90, pp1603-1625, 1985, incorporated herein by reference).

In another embodiment of the invention, the dosage form comprises a plurality of tiny pills or mini-tablets. The tiny pills or mini-tablets provide a number of individual doses for providing various time doses for achieving a sustained-release drug delivery profile over an extended period of time up to 24 hours. The tiny pills or mini-tablets may comprise a hydrophilic polymer selected from the group consisting of a polysaccharide, agar, agarose, natural gum, alkali alginate including sodium alginate, carrageenan; fucoidan, furcellaran, laminaran, hypnea, gum arabic, gum ghatti, gum karaya, gum tragacanth, locust bean gum, pectin, amylopectin, gelatin, and a hydrophilic colloid. The hydrophilic polymer may be formed into a plurality (e.g., 4 to 50) tiny pills or mini-tablet, wherein each tiny pill or mini-tablet comprises a pre-determined dose of a (+)-1-(3,4-dichlorophenyl)-3-azabicyclo[3.1.0]hexane compound or a pharmaceutically acceptable salt thereof, e.g., a dose of about 10 ng, 0.5 mg, 1 mg, 1.2 mg, 1.4 mg, 1.6 mg, 5.0 mg etc. The tiny pills and mini-tablets may further comprise a release rate-controlling wall of 0.001 up to 10 mm thickness to provide for timed release of the active compound. Representative wall forming materials include a triglyceryl ester selected from the group consisting of glyceryl tristearate, glyceryl monostearate, glyceryl dipalmitate, glyceryl laureate, glyceryl didecenoate and glyceryl tridenoate. Other wall forming materials comprise polyvinyl acetate, phthalate, methylcellulose phthalate and microporous olefins. Procedures for manufacturing tiny pills and mini-tablets are known in the art (see, e.g., U.S. Pat. Nos. 4,434,153; 4,721,613; 4,853,229; 2,996,431; 3,139,383 and 4,752,470, each incorporated herein by reference). The tiny pills and mini-tablets may further comprise a blend of particles, which may include particles of different sizes and/or release properties, and the particles may be contained in a hard gelatin or non-gelatin capsule or soft gelatin capsule.

In yet another embodiment of the invention, drug-releasing lipid matrices can be used to formulate therapeutic compositions and dosage forms comprising a (+)-1-(3,4-dichlorophenyl)-3-azabicyclo[3.1.0]hexane compound or a pharmaceutically acceptable salt thereof. In one exemplary embodiment, solid microparticles of the active compound are coated with a thin controlled release layer of a lipid (e.g., glyceryl behenate and/or glyceryl palmitostearate) as disclosed in Farah et al., U.S. Pat. No. 6,375,987 and Joachim et al., U.S. Pat. No. 6,379,700 (each incorporated herein by reference). The lipid-coated particles can optionally be compressed to form a tablet. Another controlled release lipid-based matrix material which is suitable for use in the sustained release compositions and dosage forms of the invention comprises polyglycolized glycerides, e.g., as described in Roussin et al., U.S. Pat. No. 6,171,615 (incorporated herein by reference).

In other embodiments of the invention, drug-releasing waxes can be used for producing sustained release compositions and dosage forms comprising a (+)-1-(3,4-dichlorophenyl)-3-azabicyclo[3.1.0]hexane or a pharmaceutically acceptable salt thereof. Examples of suitable sustained drug-releasing waxes include, but are not limited to, carnauba wax, candedilla wax, esparto wax, ouricury wax, hydrogenated vegetable oil, bees wax, paraffin, ozokerite, castor wax, and mixtures thereof (see, e.g., Cain et al., U.S. Pat. No. 3,402,240; Shtohryn et al. U.S. Pat. No. 4,820,523; and Walters, U.S. Pat. No. 4,421,736, each incorporated herein by reference).

In still another embodiment, osmotic delivery systems are used for sustained release delivery of a (+)-1-(3,4-dichlorophenyl)-3-azabicyclo[3.1.0]hexane compound or a pharmaceutically acceptable salt thereof (see, e.g., Verma et al., Drug Dev. Ind. Pharm., 2000, 26:695-708, incorporated herein by reference). In one exemplary embodiment, the osmotic delivery system is an OROS® system (Alza Corporation, Mountain View, Calif.) and is adapted for oral sustained release delivery of drugs (see, e.g., U.S. Pat. No. 3,845,770; and U.S. Pat. No. 3,916,899, each incorporated herein by reference).

In another embodiment of the invention, the dosage form comprises an osmotic dosage form, which comprises a semi-permeable wall that surrounds a therapeutic composition comprising a (+)-1-(3,4-dichlorophenyl)-3-azabicyclo[3.1.0]hexane compound or a pharmaceutically acceptable salt thereof. In use within a patient, the osmotic dosage form comprising a homogenous composition imbibes fluid through the semipermeable wall into the dosage form in response to the concentration gradient across the semipermeable wall. The therapeutic composition in the dosage form develops osmotic energy that causes the therapeutic composition to be administered through an exit from the dosage form over a prolonged period of time up to 24 hours (or even in some cases up to 30 hours) to provide controlled and sustained prodrug release. These delivery platforms can provide an essentially zero order delivery profile as opposed to the spiked profiles of immediate release formulations.

In alternate embodiments of the invention, the dosage form comprises another osmotic dosage form comprising a wall surrounding a compartment, the wall comprising a semipermeable polymeric composition permeable to the passage of fluid and substantially impermeable to the passage of the active compound present in the compartment, a drug-containing layer composition in the compartment, a hydrogel push layer composition in the compartment comprising an osmotic formulation for imbibing and absorbing fluid for expanding in size for pushing the (+)-1-(3,4-dichlorophenyl)-3-azabicyclo[3.1.0]hexane compound or a pharmaceutically acceptable salt thereof composition layer from the dosage form, and at least one passageway in the wall for releasing the drug composition. This osmotic system delivers the active compound by imbibing fluid through the semipermeable wall at a fluid imbibing rate determined by the permeability of the semipermeable wall and the osmotic pressure across the semipermeable wall causing the push layer to expand, thereby delivering the active compound through the exit passageway to a patient over a prolonged period of time (up to 24 or even 30 hours). The hydrogel layer composition may comprise 10 mg to 1000 mg of a hydrogel such as a member selected from the group consisting of a polyalkylene oxide of 1,000,000 to 8,000,000 which are selected from the group consisting of a polyethylene oxide of 1,000,000 weight-average molecular weight, a polyethylene oxide of 2,000,000 molecular weight, a polyethylene oxide of 4,000,000 molecular weight, a polyethylene oxide of 5,000,000 molecular weight, a polyethylene oxide of 7,000,000 molecular weight and a polypropylene oxide of the 1,000,000 to 8,000,000 weight-average molecular weight; or 10 mg to 1000 mg of an alkali carboxymethylcellulose of 10,000 to 6,000,000 weight average molecular weight, such as sodium carboxymethylcellulose or potassium carboxymethylcellulose. The hydrogel expansion layer may comprise a hydroxyalkylcellulose of 7,500 to 4,500,00 weight-average molecular weight (e.g., hydroxymethylcellulose, hydroxyethylcellulose, hydroxypropylcellulose, hydroxybutylcellulose or hydroxypentylcellulose), an osmagent, e.g., selected from the group consisting of sodium chloride, potassium chloride, potassium acid phosphate, tartaric acid, citric acid, raffinose, magnesium sulfate, magnesium chloride, urea, inositol, sucrose, glucose and sorbitol, and other agents such a hydroxypropylalkylcellulose of 9,000 to 225,000 average-number molecular weight (e.g., hydroxypropylethylcellulose, hydroxypropypentylcellulose, hydroxypropylmethylcellulose, or hydropropylbutylcellulose), ferric oxide, antioxidants (e.g., ascorbic acid, butylated hydroxyanisole, butylatedhydroxyquinone, butylhydroxyanisol, hydroxycomarin, butylated hydroxytoluene, cephalm, ethyl gallate, propyl gallate, octyl gallate, lauryl gallate, propyl-hydroxybenzoate, trihydroxybutylrophenone, dimethylphenol, dibutylphenol, vitamin E, lecithin and ethanolamine), and/or lubricants (e.g., calcium stearate, magnesium stearate, zinc stearate, magnesium oleate, calcium palmitate, sodium suberate, potassium laureate, salts of fatty acids, salts of alicyclic acids, salts of aromatic acids, stearic acid, oleic acid, palmitic acid, a mixture of a salt of a fatty, alicyclic or aromatic acid, and a fatty, alicyclic, or aromatic acid).

In the osmotic dosage forms, the semipermeable wall comprises a composition that is permeable to the passage of fluid and impermeable to passage of the (+)-1-(3,4-dichlorophenyl)-3-azabicyclo[3.1.0]hexane compound or a pharmaceutically acceptable salt thereof. The wall is nontoxic and comprises a polymer selected from the group consisting of a cellulose acylate, cellulose diacylate, cellulose triacylate, cellulose acetate, cellulose diacetate and cellulose triacetate. The wall typically comprises 75 wt % (weight percent) to 100 wt % of the cellulosic wall-forming polymer; or, the wall can comprise additionally 0.01 wt % to 80 wt % of polyethylene glycol, or 1 wt % to 25 wt % of a cellulose ether (e.g., hydroxypropylcellulose or a hydroxypropylalkycellulose such as hydroxypropylmethylcellulose). The total weight percent of all components comprising the wall is equal to 100 wt %. The internal compartment comprises the drug-containing composition alone or in layered position with an expandable hydrogel composition. The expandable hydrogel composition in the compartment increases in dimension by imbibing the fluid through the semipermeable wall, causing the hydrogel to expand and occupy space in the compartment, whereby the drug composition is pushed from the dosage form. The therapeutic layer and the expandable layer act together during the operation of the dosage form for the release of drug to a patient over time. The dosage form comprises a passageway in the wall that connects the exterior of the dosage form with the internal compartment. The osmotic powered dosage form delivers the (+)-1-(3,4-dichlorophenyl)-3-azabicyclo[3.1.0]hexane compound or a pharmaceutically acceptable salt thereof from the dosage form to the patient at a zero order rate of release over a period of up to about 24 hours. As used herein, the expression “passageway” comprises means and methods suitable for the metered release of a (+)-1-(3,4-dichlorophenyl)-3-azabicyclo[3.1.0]hexane compound or a pharmaceutically acceptable salt thereof from the compartment of an osmotic dosage form. The exit means comprises at least one passageway, including orifice, bore, aperture, pore, porous element, hollow fiber, capillary tube, channel, porous overlay, or porous element that provides for the osmotic controlled release of the active compound. The passageway includes a material that erodes or is leached from the wall in a fluid environment of use to produce at least one controlled-release dimensioned passageway. Representative materials suitable for forming a passageway, or a multiplicity of passageways comprise a leachable poly(glycolic) acid or poly(lactic) acid polymer in the wall, a gelatinous filament, poly(vinyl alcohol), leach-able polysaccharides, salts, and oxides. A pore passageway, or more than one pore passageway, can be formed by leaching a leachable compound, such as sorbitol, from the wall. The passageway possesses controlled-release dimensions, such as round, triangular, square and elliptical, for the metered release of prodrug from the dosage form. The dosage form can be constructed with one or more passageways in spaced apart relationship on a single surface or on more than one surface of the wall. The expression “fluid environment” denotes an aqueous or biological fluid as in a human patient, including the gastrointestinal tract. Passageways and equipment for forming passageways are disclosed in U.S. Pat. Nos. 3,845,770; 3,916,899; 4,063,064; 4,088,864; 4,816,263; 4,200,098; and 4,285,987 (each incorporated herein by reference).

In more detailed embodiments, a compound as disclosed herein may be encapsulated for delivery in microcapsules, microparticles, or microspheres, prepared, for example, by coacervation techniques or by interfacial polymerization, for example, hydroxymethylcellulose or gelatin-microcapsules and poly(methylmethacylate) microcapsules, respectively, in colloidal drug delivery systems (for example, liposomes, albumin microspheres, microemulsions, nano-particles and nanocapsules) or in macroemulsions.

A variety of methods is known by which a (+)-1-(3,4-dichlorophenyl)-3-azabicyclo[3.1.0]hexane compound or a pharmaceutically acceptable salt thereof can be encapsulated in the form of microparticles, for example using by encapsulating the active compound within a biocompatible, biodegradable wall-forming material (e.g., a polymer)—to provide sustained or delayed release of the active compound. In these methods, the active compound is typically dissolved, dispersed, or emulsified in a solvent containing the wall forming material. Solvent is then removed from the microparticles to form the finished microparticle product. Examples of conventional microencapsulation processes are disclosed, e.g., in U.S. Pat. Nos. 3,737,337; 4,389,330; 4,652,441; 4,917,893; 4,677,191; 4,728,721; 5,407,609; 5,650,173; 5,654,008; and 6,544,559 (each incorporated herein by reference). These documents disclose methods that can be readily implemented to prepare microparticles containing a (+)-1-(3,4-dichlorophenyl)-3-azabicyclo[3.1.0]hexane compound or a pharmaceutically acceptable salt thereof in a sustained release formulation according to the invention. As explained, for example, in U.S. Pat. No. 5,650,173, by appropriately selecting the polymeric materials, a microparticle formulation can be made in which the resulting microparticles exhibit both diffusional release and biodegradation release properties. For a diffusional mechanism of release, the active agent is released from the microparticles prior to substantial degradation of the polymer. The active agent can also be released from the microparticles as the polymeric excipient erodes. In addition, US. Pat. No. 6,596,316 (incorporated herein by reference) discloses methods for preparing microparticles having a selected release profile for fine tuning a release profile of an active agent from the microparticles.

In another embodiment of the invention, enteric-coated preparations can be used for oral sustained release administration. Preferred coating materials include polymers with a pH-dependent solubility (i.e., pH-controlled release), polymers with a slow or pH-dependent rate of swelling, dissolution or erosion (i.e., time-controlled release), polymers that are degraded by enzymes (i.e., enzyme-controlled release) and polymers that form firm layers that are destroyed by an increase in pressure (i.e., pressure-controlled release). Enteric coatings may function as a means for mediating sustained release of a (+)-1-(3,4-dichlorophenyl)-3-azabicyclo[3.1.0]hexane compound or a pharmaceutically acceptable salt thereof by providing one or more barrier layers, which may be located entirely surrounding the active compound, between layers of a multi-layer solid dosage form (see below), and/or on one or more outer surfaces of one or multiple layers of a multi-layer solid dosage form (e.g., on end faces of layers of a substantially cylindrical tablet). Such barrier layers may, for example, be composed of polymers which are either substantially or completely impermeable to water or aqueous media, or are slowly erodible in water or aqueous media or biological liquids and/or which swell in contact with water or aqueous media. Suitable polymers for use as a barrier layer include acrylates, methacrylates, copolymers of acrylic acid, celluloses and derivatives thereof such as ethylcelluloses, cellulose acetate propionate, polyethylenes and polyvinyl alcohols etc. Barrier layers comprising polymers which swell in contact with water or aqueous media may swell to such an extent that the swollen layer forms a relatively large swollen mass, the size of which delays its immediate discharge from the stomach into the intestine. The barrier layer may itself contain active material content, for example the barrier layer may be a slow or delayed release layer. Barrier layers may typically have an individual thickness of 10 microns up to 2 mm. Suitable polymers for barrier layers which are relatively impermeable to water include the Methocel™ series of polymers, used singly or combined, and Ethocel™ polymers. Such polymers may suitably be used in combination with a plasticizer such as hydrogenated castor oil. The barrier layer may also include conventional binders, fillers, lubricants and compression acids etc. such as Polyvidon K30 (trade mark), magnesium stearate, and silicon dioxide.

Additional enteric coating materials for mediating sustained release of a (+)-1-(3,4-dichlorophenyl)-3-azabicyclo[3.1.0]hexane or a pharmaceutically acceptable salt thereof include coatings in the form of polymeric membranes, which may be semipermeable, porous, or asymmetric membranes (see, e.g., U.S. Pat. No. 6,706,283, incorporated herein by reference). Coatings of these and other types for use within the invention may also comprise at least one delivery port, or pores, in the coating, e.g., formed by laser drilling or erosion of a plug of water-soluble material. Other useful coatings within the invention including coatings that rupture in an environment of use (e.g., a gastrointestinal compartment) to form a site of release or delivery port. Exemplary coatings within these and other embodiments of the invention include poly(acrylic) acids and esters; poly(methacrylic) acids and esters; copolymers of poly(acrylic) and poly(methacrylic) acids and esters; cellulose esters; cellulose ethers; and cellulose ester/ethers.

Additional coating materials for use in constructing solid dosage forms to mediate sustained release of a (+)-1-(3,4-dichlorophenyl)-3-azabicyclo[3.1.0]hexane compound or a pharmaceutically acceptable salt thereof include, but are not limited to, polyethylene glycol, polypropylene glycol, copolymers of polyethylene glycol and polypropylene glycol, poly(vinylpyrrolidone), ethyl cellulose, hydroxyethyl cellulose, hydroxypropyl cellulose, carboxymethyl cellulose, carboxymethylethyl cellulose, starch, dextran, dextrin, chitosan, collagen, gelatin, bromelain, cellulose acetate, unplasticized cellulose acetate, plasticized cellulose acetate, reinforced cellulose acetate, cellulose acetate phthalate, cellulose acetate trimellitate, hydroxypropylmethylcellulose, hydroxypropylmethyl-cellulose phthalate, hydroxypropylmethylcellulose acetate succinate, hydroxypropylmethylcellulose acetate trimellitate, cellulose nitrate, cellulose diacetate, cellulose triacetate, agar acetate, amylose triacetate, beta glucan acetate, beta glucan triacetate, acetaldehyde dimethyl acetate, cellulose acetate ethyl carbamate, cellulose acetate phthalate, cellulose acetate methyl carbamate, cellulose acetate succinate, cellulose acetate dimethaminoacetate, cellulose acetate ethyl carbonate, cellulose acetate chloroacetate, cellulose acetate ethyl oxalate, cellulose acetate methyl sulfonate, cellulose acetate butyl sulfonate, cellulose acetate propionate, cellulose acetate p-toluene sulfonate, triacetate of locust gum bean, cellulose acetate with acetylated hydroxyethyl cellulose, hydroxlated ethylene-vinylacetate, cellulose acetate butyrate, polyalkenes, polyethers, polysulfones, polyethersulfones, polystyrenes, polyvinyl halides, polyvinyl esters and ethers, natural waxes and synthetic waxes.

In additional embodiments of the invention, sustained release of a (+)-1-(3,4-dichlorophenyl)-3-azabicyclo[3.1.0]hexane compound or a pharmaceutically acceptable salt thereof is provided by formulating the active compound in a dosage form comprising a multi-layer tablet or other multi-layer or multi-component dosage form. In exemplary embodiments, the active compound is formulated in layered tablets, for example having a first layer which is an immediate release layer and a second layer which is a slow release layer. Other multi-layered dosage forms of the invention may comprise a plurality of layers of compressed active ingredient having variable (i.e., selectable) release properties selected from immediate, extended and/or delayed release mechanisms. Multi-layered tablet technologies useful to produce sustained release dosage forms of a (+)-1-(3,4-dichlorophenyl)-3-azabicyclo[3.1.0]hexane compound or a pharmaceutically acceptable salt thereof are described, for example, in International Publications WO 95/20946; WO 94/06416; and WO 98/05305 (each incorporated herein by reference). Other multi-component dosage forms for providing sustained delivery of a (+)-1-(3,4-dichlorophenyl)-3-azabicyclo[3.1.0]hexane compound or a pharmaceutically acceptable salt thereof include tablet formulations having a core containing the active compound coated with a release retarding agent and surrounded by an outer casing layer (optionally containing the active compound) (see, e.g., International Publication WO 95/28148, incorporated herein by reference). The release retarding agent is an enteric coating, so that there is an immediate release of the contents of the outer core, followed by a second phase from the core which is delayed until the core reaches the intestine. Additionally, International Publication WO 96/04908 (incorporated herein by reference) describes tablet formulations which comprise an active agent in a matrix, for immediate release, and granules in a delayed release form comprising the active agent. Such granules are coated with an enteric coating, so release is delayed until the granules reach the intestine. International Publication WO 96/04908 (incorporated herein by reference) describes delayed or sustained release formulations formed from granules which have a core comprising an active agent, surrounded by a layer comprising the active agent.

Another useful multi-component (bi-layer tablet) dosage form for sustained delivery of a (+)-1-(3,4-dichlorophenyl)-3-azabicyclo[3.1.0]hexane compound or a pharmaceutically acceptable salt thereof is described in U.S. Pat. No. 6,878,386 (incorporated herein by reference). Briefly, the bilayer tablet comprises an immediate release and a slow release layer, optionally with a coating layer. The immediate release layer may be, for example, a layer which disintegrates immediately or rapidly and has a composition similar to that of known tablets which disintegrate immediately or rapidly. An alternative type of immediate release layer may be a swellable layer having a composition which incorporates polymeric materials which swell immediately and extensively in contact with water or aqueous media, to form a water permeable but relatively large swollen mass. Active material content may be immediately leached out of this mass. The slow release layer may have a composition comprising a (+)-1-(3,4-dichlorophenyl)-3-azabicyclo[3.1.0]hexane compound or a pharmaceutically acceptable salt thereof with a release retarding vehicle, matrix, binder, coating, or excipient which allows for slow release of the active compound. Suitable release retarding excipients include pH sensitive polymers, for instance polymers based upon methacrylic acid copolymers, which may be used either alone or with a plasticiser; release-retarding polymers which have a high degree of swelling in contact with water or aqueous media such as the stomach contents; polymeric materials which form a gel on contact with water or aqueous media; and polymeric materials which have both swelling and gelling characteristics in contact with water or aqueous media. Release retarding polymers which have a high degree of swelling include, inter alia, cross-linked sodium carboxymethylcellulose, cross-linked hydroxypropylcellulose, high-molecular weight hydroxypropylmethylcellulose, carboxymethylamide, potassium methacrylatedivinylbenzene co-polymer, polymethylmethacrylate, cross-linked polyvinylpyrrolidone, high-molecular weight polyvinylalcohols etc. Release retarding gellable polymers include methylcellulose, carboxymethylcellulose, low-molecular weight hydroxypropylmethylcellulose, low-molecular weight polyvinylalcohols, polyoxyethyleneglycols, non-cross linked polyvinylpyrrolidone, xanthan gum etc. Release retarding polymers simultaneously possessing swelling and gelling properties include medium-viscosity hydroxypropylmethylcellulose and medium-viscosity polyvinylalcohols. An exemplary release-retarding polymer is xanthan gum, in particular a fine mesh grade of xanthan gum, preferably pharmaceutical grade xanthan gum, 200 mesh, for instance the product Xantural 75 (also known as Keltrol CR™ Monsanto, 800 N Lindbergh Blvd, St Louis, Mo. 63167, USA). Xanthan gum is a polysaccharide which upon hydration forms a viscous gel layer around the tablet through which the active has to diffuse. It has been shown that the smaller the particle size, the slower the release rate. In addition, the rate of release of active compound is dependent upon the amount of xanthan gum used and can be adjusted to give the desired profile. Examples of other polymers which may be used within these aspects of the invention include Methocel K4M™, Methocel E5™, Methocel E5O™, Methocel E4M™, Methocel K15M™ and Methocel K100M™. Other known release-retarding polymers which may be incorporated within this and other embodiments of the invention to provide a sustained release composition or dosage form of a (+)-1-(3,4-dichlorophenyl)-3-azabicyclo[3.1.0]hexane compound or a pharmaceutically acceptable salt thereof include, hydrocolloids such as natural or synthetic gums, cellulose derivatives other than those listed above, carbohydrate-based substances such as acacia, gum tragacanth, locust bean gum, guar gum, agar, pectin, carrageenan, soluble and insoluble alginates, carboxypolymethylene, casein, zein, and the like, and proteinaceous substances such as gelatin.

Within other embodiments of the invention, a sustained release delivery device or system is placed in the subject in proximity of the target of the active compound, thus requiring only a fraction of the systemic dose (see, e.g., Goodson, in “Medical Applications of Controlled Release,” supra, vol. 2, pp. 115-138, 1984; and Langer, 1990, Science 249:1527-1533, each incorporated herein by reference). In other embodiments, an oral sustained release pump may be used (see, e.g., Langer, supra; Sefton, 1987, CRC Crit. Ref. Biomed. Eng. 14:201; and Saudek et al., 1989, N. Engl. J. Med. 321:574, each incorporated herein by reference).

The pharmaceutical compositions and dosage forms used in the current invention will typically be provided for administration in a sterile or readily sterilizable, biologically inert, and easily administered form.

In other embodiments the invention provides pharmaceutical kits for reducing symptoms in a human subject suffering from a disorder affected by monoamine neurotransmitters, including depression. The kits comprise a (+)-1-(3,4-dichlorophenyl)-3-azabicyclo[3.1.0]hexane compound or a pharmaceutically acceptable salt thereof in an effective amount, and a container means for containing the (+)-1-(3,4-dichlorophenyl)-3-azabicyclo[3.1.0]hexane compound or a pharmaceutically acceptable salt thereof for coordinate administration to the said subject (for example a container, divided bottle, or divided foil pack). The container means can include a package bearing a label or insert that provides instructions for multiple uses of the kit contents to treat the disorder and reduce symptoms in the subject. In more detailed embodiments, the (+)-1-(3,4-dichlorophenyl)-3-azabicyclo[3.1.0]hexane compound or a pharmaceutically acceptable salt thereof is admixed or co-formulated in a single, combined dosage form, for example a liquid or solid oral dosage form. In alternate embodiments, the (+)-1-(3,4-dichlorophenyl)-3-azabicyclo[3.1.0]hexane compound or a pharmaceutically acceptable salt thereof is contained in the kit in separate dosage forms for coordinate administration. An example of such a kit is a so-called blister pack. Blister packs are well-known in the packaging industry and are widely used for the packaging of pharmaceutical dosage forms (tablets, capsules and the like).

Unless the context clearly requires otherwise, throughout the description and the claims, the words “comprise,” “comprising,” and the like are to be construed in an inclusive sense as opposed to an exclusive or exhaustive sense; that is to say; in the sense of “including, but not limited to.” Words using the singular or plural number also include the plural or singular number respectively. Additionally, the words “herein,” “above,” “below” and words of similar import, when used in this application, refer to this application as a whole and not to any particular portions of this application. When the claims use the word “or” in reference to a list of two or more items, that word covers all of the following interpretations of the word: any of the items in the list, all of the items in the list and any combination of the items in the list.

It is to be understood that this invention is not limited to the particular formulations, process steps, and materials disclosed herein as such formulations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

The following examples illustrate certain aspects of the invention, but are not intended to limit in any manner the scope of the invention.

### EXAMPLE I

### The Effect of (+)-1-(3,4-dichlorophenyl)-3-azabicyclo[3.1.0]hexane on the Consumption of Ethanol by Alcohol Preferring Rats

The efficacy of (+)-1-(3,4-dichlorophenyl)-3-azabicyclo[3.1.0]hexane to reduce alcohol-drinking behavior was evaluated in the genetically selected alcohol-preferring (P) rat, an animal model for human alcohol abuse. The effects of (+)-1-(3,4-dichlorophenyl)-3-azabicyclo[3.1.0]hexane on P rats were examined in a binge operant responding experiment.

P rats were obtained from the Alcohol Research Center at Indiana University School of Medicine. Male P rats were used in all experiments, and were approximately 4-5 months of age and weighed between 420 and 580 g at the outset. Animals were housed individually at an ambient temperature of 21° C., with a normal 12 hour light/dark cycle. All rats were provided ad libitum access to food and water, except during training for operant self-administration studies, during which rats were fluid deprived 23 hr daily during the first 5 days of the training phase. Thereafter, these animals were maintained on ad libitum food and water. All training and experimental sessions for all subjects took place between 9 a.m. and 5 p.m.

The P rats were orally admininstered (PO) (+)-1-(3,4-dichlorophenyl)-3-azabicyclo[3.1.0]hexane in deionized water using feed tubes and an injection volume of 1 ml/kg. Deionized water was administered as the control injection.

Behavioral testing was conducted in standard operant chambers (Coulbourn Instruments, Allentown, Pa.) equipped with two removable levers and two dipper fluid delivery systems enclosed in sound-attenuated cubicles as previously described (Cook et al., 2004). All dipper presentations provided a 1.5-sec access to a 0.1-ml dipper, followed by a 3-sec time out period. Above each lever, three stimulus lights (red, green and yellow) were present, and a stimulus delivery/reinforcer was indicated by illumination of the middle (green) stimulus light. Responses and reinforcements were recorded and controlled using the 4.0 Coulbourn L2T2 operant software package. Operant sessions were 90 minutes.

Rats were trained to lever press for EtOH (10% v/v) using a modified version of the sucrose fading-technique, as previously described (Foster et al., 2004). In brief, for all training protocols, animals were water-deprived on the first 5 days of training, using a 23 hr fluid deprivation schedule to facilitate lever-pressing. Initially, the rats lever pressed for sucrose (3% w/v) under a Fixed-Ratio 1 (FR1) schedule for about 5-7 days. Rats were then divided into sucrose and alcohol reinforcement groups. Rats belonging to the sucrose group were trained and subsequently stabilized on a fixed-ratio (FR) 4 schedule of reinforcement on both the right and left levers. Rats belonging to the alcohol group underwent the sucrose fading procedure wherein they subsequently responded under a FR4 schedule for EtOH (10% v/v) on both the right and left levers. Stabilization for both the sucrose and alcohol reinforcers were defined as having daily responses within±20% of the average responses for five consecutive days.

For the alcohol and sucrose maintained responding studies, (+)-1-(3,4-dichlorophenyl)-3-azabicyclo[3.1.0]hexane [3.1, 6.3, 12.5, 25, or 50 mg/kg] was orally administered 25 min before each P rat was placed in an operant chamber to allow for optimal absorption and CNS distribution. The rats were then observed in the operant chambers for 90 minutes. Seven to ten rats were evaluated with each concentration of (+)-1-(3,4-dichlorophenyl)-3-azabicyclo[3.1.0]hexane [3.1-50 mg/kg].

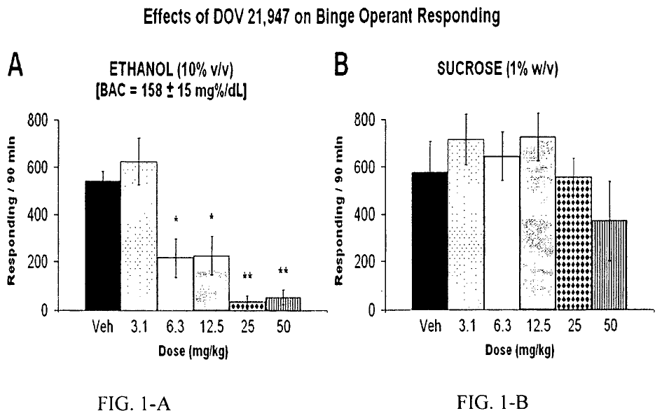

The results of the binge operant responding experiment are shown in FIG. 1. The alcohol maintained P rats had a pharmacologically relevant blood alcohol concentration (BAC) of 158±mg %/dL. The graph in FIG. 1A demonstrates a dose response for (+)-1-(3,4-dichlorophenyl)-3-azabicyclo[3.1.0]hexane in P rats. The graph in FIG. 1B depicts the lack of effect of (+)-1-(3,4-dichlorophenyl)-3-azabicyclo[3.1.0]hexane on P rats maintained on sucrose water. These results demonstrate that the effect of (+)-1-(3,4-dichlorophenyl)-3-azabicyclo[3.1.0]hexane on P rat alcohol consumption was not due to an anhedonic state or a non-specific effect.

### EXAMPLE II

### The Effects of (+)-1-(3,4-dichlorophenyl)-3-azabicyclo[3.1.0]hexane on the Consumption of Ethanol by High-Alcohol Preferring Mice

The efficacy of (+)-1-(3,4-dichlorophenyl)-3-azabicyclo[3.1.0]hexane to reduce alcohol-drinking behavior was evaluated in the genetically selected high-alcohol preferring (HAP) mouse, an animal model for human alcohol abuse. The effects of (+)-1-(3,4-dichlorophenyl)-3-azabicyclo[3.1.0]hexane on HAP mice were examined in a binge operant responding experiment, using standard protocols.

HAP mice were obtained from the Alcohol Research Center at Indiana University School of Medicine. Animals were housed individually at an ambient temperature of 21° C., with a normal 12 hour light/dark cycle. All mice were provided ad libitum access to food and water, except during training for operant self-administration studies, during which mice were fluid deprived daily during the first 5 days of the training phase. Thereafter, these animals were maintained on ad libitum food and water.

The HAP mice were intraperitoneally injected with (+)-1-(3,4-dichlorophenyl)-3-azabicyclo[3.1.0]hexane in a saline solution. The saline solution was administered as the control injection.

Behavioral testing was conducted in standard operant chambers (Coulbourn Instruments, Allentown, Pa.) equipped with two removable levers and two dipper fluid delivery systems enclosed in sound-attenuated cubicles as previously described (Cook et al., 2004). All dipper presentations provided a 1.5-sec access to a 0.1-ml dipper, followed by a 3-sec time out period. Above each lever, three stimulus lights (red, green and yellow) were present, and a stimulus delivery/reinforcer was indicated by illumination of the middle (green) stimulus light. Responses and reinforcements were recorded and controlled using the 4.0 Coulbourn L2T2 operant software package. Operant sessions were 90 minutes.

HAP mice were trained to lever press for EtOH (10% v/v) using a modified version of the sucrose fading-technique, as previously described (Foster et al., 2004). In brief, for all training protocols, animals were water-deprived on the first 5 days of training, using a fluid deprivation schedule to facilitate lever-pressing. Initially, the mice lever pressed for sucrose (3% w/v) under a Fixed-Ratio 1 (FR1) schedule for about 5-7 days. Mice were then divided into sucrose and alcohol reinforcement groups. HAP mice belonging to the sucrose group were trained and subsequently stabilized on a fixed-ratio (FR) 4 schedule of reinforcement on both the right and left levers. HAP mice belonging to the alcohol group underwent the sucrose fading procedure wherein they subsequently responded under a FR4 schedule for EtOH (10% v/v) on both the right and left levers. Stabilization for both the sucrose and alcohol reinforcers were defined as having daily responses within±20% of the average responses for five consecutive days.

For the alcohol and sucrose maintained responding studies, (+)-1-(3,4-dichlorophenyl)-3-azabicyclo[3.1.0]hexane [25, 50, or 75 mg/kg] was injected into each HAP mouse, followed by immediate placement in an operant chamber. The mice were then observed in the operant chambers for 90 minutes. Six HAP mice were evaluated with each concentration of (+)-1-(3,4-dichlorophenyl)-3-azabicyclo[3.1.0]hexane [25, 50, or 75 mg/kg].

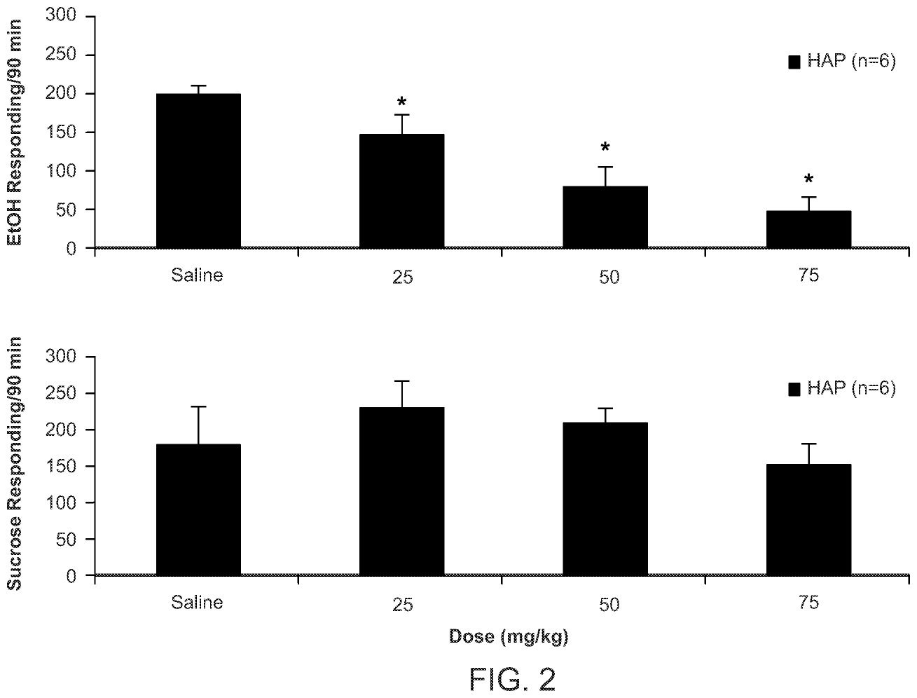

The results of the binge operant responding experiment are shown in FIG. 2. The ethanol responding graph in FIG. 2 demonstrates a dose response for (+)-1-(3,4-dichlorophenyl)-3-azabicyclo[3.1.0]hexane in HAP mice. The control sucrose responding graph in FIG. 2 depicts the lack of effect of (+)-1-(3,4-dichlorophenyl)-3-azabicyclo[3.1.0]hexane on HAP mice maintained on sucrose water. These results indicate that (+)-1-(3,4-dichlorophenyl)-3-azabicyclo[3.1.0]hexane had specific effects on HAP mouse alcohol consumption.

All publications and patents cited herein are incorporated herein by reference for the purpose of describing and disclosing, for example, the materials and methodologies that are described in the publications, which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

### REFERENCES

Astrup A, Madsbad S, Breum L, Jensen T J, Kroustrup J P, Larsen T M. Effect of Tesofensine on Bodyweight Loss, Body Composition, and Quality of Life in Patients: a Randomised, Double-Blind, Placebo-Controlled Trial. Lancet 2008, 372:1906-13.
DeLorenzo C, Lichenstein S, Schaefer K, Dunn J, Marshall R, Organisak L, Kharidia J, Robertson B, Mann J J, Parsey R V. SEP-225289 Serotonin and Dopamine Transporter Occupancy: a PET Study. J. Nucl. Med. 2011, 52:1150-1155.
FDA New Release, Oct. 8, 2010, http://www.fda.gov/NewsEvents/Newsroom/PressAnnouncements/2010/ucm228812.htm.
Graff, Ole et al. Results of two double blind Placebo and Active-controlled Studies of GSK372475, a Triple Monoamine Reuptake Inhibitor, in the Treatment of Major Depressive Disorder. (ACNP 2009)
Guha M, Heier A, Price S, Bielenstein M, Caccese R G, Heathcote D I, Simpson T R, Stong D B, Bodes E. Assessment of Biomarkers of Drug-induced Kidney Injury in Cynomolgus Monkeys Treated with a Triple Re-uptake Inhibitor. Toxicol. Sci. 2011, 120:269-83.
James W P, Caterson I D, Coutinho W, Finer N, Van Gaal L F, Maggioni A P, Torp-Pedersen C, Sharma A M, Shepherd G M, Rode R A, Renz C L. Effect of sibutramine on cardiovascular outcomes in overweight and obese subjects. N Engl J Med. 2010, 363:905-17.
Rascol O, Poewe W, Lees A, Aristin M, Salin L, Juhel N, Waldhause L, Schindler T. Tesofensine (NS2330), a Monoamine Reuptake Inhibior, in Patients with Advanced Parkinson Disease and Motor Fluctuations. Arch. Neurol 2008, 65:577-583.
Sepracor Press Release, Jul. 1, 2009 http://www.fiercebiotech.com/press-releases/sepracor-provides-update-clinical-trials-sep-225289-and-lunesta-r-pediatrics.
Skolnick M, Popik P, Janowsky A, Beer B, Lippa A: Antidepressant-like actions of DOV 21,947: A triple reuptake inhibitor. European Journal of Pharmacology 2003 461:99-104
Wilens T E, Klint T, Adler L, West S, Wesnes K, Graff O, Mikkelsen B. A Randomized Controlled Trial of a Novel Mixed Monamine Reuptake Inhibitor in Adults with ADHD. Behavioral and Brain Functions 2008, 4:24.

What is claimed is:

1. A method for treating or preventing an alcohol-related or addictive disorder alleviated comprising administering to a patient in need of such treatment or prevention an effective amount of (+)-1-(3,4-dichlorophenyl)-3-azabicyclo[3.1.0]hexane or a pharmaceutically acceptable salt thereof, each being substantially free of its corresponding (−)-enantiomer.

2. The method according to claim 1, wherein the (+)-1-(3,4-dichlorophenyl)-3-azabicyclo[3.1.0]hexane or pharmaceutically acceptable salt thereof has no more than about 2% w/w of the corresponding (−)-enantiomer.

3. The method according to claim 1, wherein the (+)-1-(3,4-dichlorophenyl)-3-azabicyclo[3.1.0]hexane or pharmaceutically acceptable salt thereof has no more than about 1% w/w of the corresponding (−)-enantiomer.

4. The method according to claim 1, wherein said (+)-1-(3,4-dichlorophenyl)-3-azabicyclo[3.1.0]hexane or a pharmaceutically acceptable salt thereof is effective to reduce alcohol consumption by said patient in comparison to a control subject that does not receive said (+)-1-(3,4-dichlorophenyl)-3-azabicyclo[3.1.0]hexane or a pharmaceutically acceptable salt thereof.

5. The method according to claim 1, wherein said alcohol-related disorder is selected from the group consisting of Alcohol-Induced Psychotic Disorder, with delusions; Alcohol Abuse; Alcohol Intoxication; Alcohol Withdrawal; Alcohol Intoxication Delirium; Alcohol Withdrawal Delirium; Alcohol-Induced Persisting Dementia; Alcohol-Induced Persisting Amnestic Disorder; Alcohol Dependence; Alcohol-Induced Psychotic Disorder, with hallucinations; Alcohol-Induced Mood Disorder; Alcohol-Induced Anxiety Disorder; Alcohol-Induced Sexual Dysfunction; Alcohol-Induced Sleep Disorders; Alcohol-Related Disorders not otherwise specified (NOS); and Alcohol Intoxication.

6. The method according to claim 1, wherein the addictive disorder is selected from the group consisting of eating disorders, impulse control disorders, nicotine-related disorders, amphetamine-related disorders, cannabis-related disorders, cocaine-related disorders, hallucinogen-use disorders, inhalant-related disorders, and opioid-related disorders.

7. The method according to claim 1 further comprising administering another therapeutic agent.

8. The method according to claim 7, wherein the wherein the other therapeutic agent is an anti-alcohol agent.

9. The method according to claim 8 wherein said anti-alcohol agent is selected from the group consisting of disulfram, naltrexone, acamprosate, ondansetron, sertraline, galanthamine, nalmefene, naloxone, desoxypeganine, benzodiapenes, neuroleptics, risperidone, remonabant, trazaodone, topiramate, and aripiprazole.

10. The method according to claim 7, wherein the wherein the other therapeutic agent is an anti-nicotine agent.

11. The method according to claim 7, wherein the wherein the other therapeutic agent is an anti-opiate agent.

12. The method according to claim 7, wherein the wherein the other therapeutic agent is an anti-cocaine agent.

13. The method according to claim 7, wherein the wherein the other therapeutic agent is an anti-depressant.

14. The method according to claim 7, wherein the wherein the other therapeutic agent is an anti-psychotic drug.

15. The method according to claim 7, wherein the wherein the other therapeutic agent is an anxiolytic agent.

16. The method according to claim 7, wherein the wherein the other therapeutic agent is an anti-alcohol agent.

17. A method for treating or preventing ethanol consumption comprising administering to a patient in need of such treatment or prevention an effective amount (+)-1-(3,4-dichlorophenyl)-3-azabicyclo[3.1.0]hexane or a pharmaceutically acceptable salt thereof, each being substantially free of its corresponding (−)-enantiomer

18. The method according to claim 17, wherein the (+)-1-(3,4-dichlorophenyl)-3-azabicyclo[3.1.0]hexane or pharmaceutically acceptable salt thereof has no more than about 2% w/w of the corresponding (−)-enantiomer.

19. The method according to claim 17, wherein the (+)-1-(3,4-dichlorophenyl)-3-azabicyclo[3.1.0]hexane or pharmaceutically acceptable salt thereof has no more than about 1% w/w of the corresponding (−)-enantiomer.

20. The method according to claim 17 further comprising administering another therapeutic agent.

21. The method according to claim 20, wherein the wherein the other therapeutic agent is an anti-alcohol agent.

22. The method according to claim 21 wherein said anti-alcohol agent is selected from the group consisting of disulfram, naltrexone, acamprosate, ondansetron, sertraline, galanthamine, nalmefene, naloxone, desoxypeganine, benzodiapenes, neuroleptics, risperidone, remonabant, trazaodone, topiramate, and aripiprazole.

23. A method for treating or preventing an alcohol-related or addictive disorder alleviated comprising administering to a patient in need of such treatment or prevention an effective amount of unbalanced triple reuptake inhibitor having a serotonin-norepinephrine-dopamine reuptake inhibition ratio ranging from about 1-2:1-3:4-12 or a pharmaceutically acceptable salt thereof.
